(19) United States
(12) Patent Application Publication
(10) Pub. No.: US 2025/0263671 A1
(43) Pub. Date: Aug. 21, 2025

(54) NEURON-INNERVATED ASSEMBLOIDS AND METHODS OF MAKING THE SAME

(71) Applicant: University of Georgia Research Foundation, Inc., Athens, GA (US)

(72) Inventors: Nadja Zeltner, Athens, GA (US); Hsueh-Fu Wu, Fort Lee, NJ (US)

(21) Appl. No.: 19/054,529

(22) Filed: Feb. 14, 2025

Related U.S. Application Data
(60) Provisional application No. 63/553,808, filed on Feb. 15, 2024.

(51) Int. Cl. C12N 5/071; G01N 33/50

(52) U.S. Cl. CPC C12N 5/0697; G01N 33/5088; C12N 2502/081; C12N 2502/1329; C12N 2513/00; C12N 2527/00

(57) ABSTRACT

Methods of making assembloids, the assembloids, compositions made from and/or including assembloids, and methods of using the foregoing are provided. Methods of making assembloids typically include combining dissociated neuron progenitor cells with dissociated tissue progenitor cells and culturing them under free floating 3D culture conditions suitable for the neuron progenitor cells and tissue progenitor cells to form one or more assembloids. In some forms, the assembloids are cultured under suitable conditions and duration for the neuron progenitor cells and tissue progenitor cells to mature. The neuron progenitor cells can be, for example, sympathetic neuron progenitor cells, parasympathetic neuron progenitor cells, and/or sensory neuron progenitor cells. The tissue progenitor cells can be, for example, heart, lung, kidney, liver, salivary gland, skin, and/or gastro-intestinal progenitor cells. Assembloids are also provided, as are compositions including assembloids, and conditioned media formed from assembloids.

### CROSS-REFERENCE TO RELATED APPLICATIONS

This application claims the benefit of and priority to U.S. Provisional Application No. 63/553,808, filed Feb. 15, 2024, which is hereby incorporated herein by reference in its entirety.

### STATEMENT REGARDING FEDERALLY SPONSORED RESEARCH OR DEVELOPMENT

This invention was made with government support under NS114567-01A1 awarded by the National Institutes of Health. The government has certain rights in the invention.

### FIELD OF THE INVENTION

The field of the invention generally relates to compositions and methods of making innervated organoids.

### BACKGROUND OF THE INVENTION

Human pluripotent stem cell (hPSC)-based 3D organoid strategies have become powerful tools to study human organ development, for disease modeling and drug discovery over the past years. Assembloids result from the integration of multiple organoids or combination of organoids with other cell types¹⁻³. Organoids of various organs have been described, including brain, kidney, heart, lung, liver and more⁴⁻⁸. Most organs of the body, except the brain, are innervated and thus regulated by the autonomic nervous system. Specifically, the sympathetic nervous system is responsible for activation of most organ's cell types. A key challenge in the organoid field is the lack of innervation of organoids, despite neural regulation being vital for organ and thus organoid development, integrity, and function.

Innervation of peripheral tissue organoids has been reported so far. Intestinal organoids were innervated via mixing of vagal neural crest cells into human intestinal organoids (HIOs)⁹. This study resulted in organoids containing functional enteric neurons and glia. However, the enteric nervous system is specific to the gastrointestinal tract and thus cannot easily be adapted for innervation of other organ type organoids, thus it lacks versatility. Neuromesodermal progenitor (NMP)-derived organoids were reported, where the NMP gave rise to both skeletal muscle and motor neurons from the same progenitor within the same cultures¹⁰, as an one-pot differentiation¹¹. This study, however, is not easily adaptable to other organ type organoids, if a common neural-tissue specific progenitor is not readily available. For example, it would be challenging to differentiate symN-innervated lung organoids, since there is no known common lung and symN progenitor. Also, one-pot differentiation protocols make it difficult to perform cell type-specific, genetic manipulations within the organoids. Schneider et al., showed exciting results of bioengineered heart organoids with assembled autonomic organoids creating an innervated cardiac assembloid. This elegant study requires advanced bioengineering capabilities and isometric force instrumentation for analysis¹². Such approaches are not easily accessible to many researchers. Therefore, there remains a need for improved compositions and methods for making innervated organoids and assembloids.

Thus, it is an object of the invention to provide simple, highly versatile strategies for innervation of organoids.

It is further object of the invention to provide strategies that may be adapted to most organ's organoid type and neuron types.

### SUMMARY OF THE INVENTION

Methods of making assembloids, the assembloids, compositions made from and/or including assembloids, and methods of using the foregoing are provided. Methods of making assembloids typically include combining dissociated neuron progenitor cells with dissociated tissue progenitor cells and culturing them under free floating 3D culture conditions suitable for the neuron progenitor cells and tissue progenitor cells to form one or more assembloids.

In some forms, the assembloids are cultured under suitable conditions and duration for the neuron progenitor cells and tissue progenitor cells to mature. Maturation can include, for example, expression of one or more markers; presence of one or more structures and/or ultrastructures; and/or one or more functionalities consistent with the corresponding mature tissue or system in vivo.

In some forms, the method is carried out free from special instruments and/or scaffolds, optionally wherein the special instrument and/or scaffold is or includes a bioprinter, hydrogel, and/or organ-mimicking scaffold.

In some forms, the neuron progenitor cells and tissue progenitor cells are mixed and/or cultured using a shaker, such as an orbital shaker.

The neuron progenitor cells can be, for example, sympathetic neuron progenitor cells, parasympathetic neuron progenitor cells, and/or sensory neuron progenitor cells, optionally wherein the sensory neuron progenitor cells are nociceptors, mechanoreceptors, and/or proprioceptors. The tissue progenitor cells can be, for example, heart, lung, kidney, liver, salivary gland, skin, and/or gastro-intestinal progenitor cells.

For example, in some forms, the neuron progenitor cells are symNblast sympathetic neuron progenitor cells prepared from human pluripotent stem cells. In some forms, the tissue progenitor cells are cardiomyocytes, e.g., 7 day cardiomyocytes prepared from human pluripotent stem cells.

Assembloid formed according to the method are also provided.

For example, 3D assembloids having neurons innervating a second or more tissues are provided.

In some forms, neurons include sympathetic neurons, parasympathetic neurons, and/or sensory neurons, optionally wherein the sensory neurons are nociceptors, mechanoreceptors, and/or proprioceptors. In some forms, the second or more tissues include heart, lung, kidney, liver, salivary gland, skin, and/or gastro-intestinal cells.

In a particular form, the assembloids include sympathetic neurons and cardiac cells.

Compositions including assembloids are also provided. Such compositions can include, for example, culture media.

Conditioned media formed by culturing assembloid(s) is also provided.

Methods of using the assembloids are also provided. For example, a method of determining the effect of a compound can include measuring one or more characteristic(s) of cultured assembloid(s) a first time, contacting the assembloid(s) with the compound, and measuring the characteristic(s) of the cultured assembloid(s) a second time. Characteristics can include, for example, expression of molecular and/or biochemical markers, and/or assembloid morphology, physiology, function, and/or activity. In some forms, a plurality of compounds are separately contacted with a plurality of separately cultured assembloids, optionally wherein the assembloids are separately cultured in a multiwell plate or dish.

### BRIEF DESCRIPTION OF THE DRAWINGS

FIGS. 1A-1N illustrate assembly of hSCAs. FIG. 1A is a schematic illustration of symNblast and symN differentiation from hPSCs. 2D: attached cell culture. 3D: suspending spheroid culture. FIG. 1B is a PCA plot showing the differentiation trajectory of symN differentiation. FIG. 1C Top: Schematic illustration of cardiac progenitor differentiation. Bottom: RT-qPCR analysis of day 7 cardiac progenitors. n=4 biological replicates. FIG. 1D is a schematic illustration of hSCA assembly and differentiation. Representative right field images of hSCA growth among time. FIG. 1E is a graph showing quantification of hSCA size overtime. n=3 biological replicates. FIG. 1F is a heatmap of RT-qPCR analysis of wk1 and wk5 hSCAs for Ki67. n=3 biological replicates. FIG. 1G is a line graph showing hSCA beating analysis overtime using the ImageJ Time Series Analyzer. n=6 biological replicates. FIG. 1H is a representative bright field image of wk5 hSCAs. FIG. 1I is a representative whole mount image of wk5 hSCAs for PRPH. FIG. 1J is a representative whole mount image of wk5 hSCAs for PHOX2B::GFP reporter. FIG. 1K is a representative cryosection image of wk5 hSCAs for a-actinin. FIG. 1L is a representative cryosection image of wk5 hSCAs for cTnT. FIG. 1M is a representative cryosection image of wk5 hSCAs for EF1::RFP reporter. FIG. 1N is a bar graph showing RT-qPCR analysis of wk5 hSCAs for symN and cardiac development markers. Error bars represent SEM. Scale bars represent 200 μm.

FIGS. 2A-2J illustrate hSCAs are self-organized and display features of maturation. FIG. 2A is bar graphs of RT-qPCR analysis of wk5 hSCAs for cardiac maturity markers. FIG. 2B is a representative TEM images with yellow arrows indicating the myofiber, Z-line, and intercalated disc in hSCAs. Scale bars represent 200 nm. FIG. 2C is a bar graph showing RT-qPCR analysis of wk5 hSCAs for cardiac T-tubule markers. FIG. 2D is a series of representative TEM images with yellow arrows indicating the T-tubules in hSCAs. Scale bar represents 200 nm. FIG. 2E is a series of representative cryosection image of wk5 hSCAs which were stained for WGA-488 and cTnT to label T-tubules. Scale bars represent 200 μm, and 50 μm in the yellow dashed rectangle. FIG. 2F is a graph showing quantification for the percentage of hSCAs with cavity structures. n=7 biological replicates. FIG. 2G is a representative cryosection image of wk5 hSCAs with DAPI showing the cavity structures. Scale bar represents 200 μm. FIG. 2H is a representative cryosection image of wk5 hSCAs for epicardial marker WT1 and endocardial marker NFATC1. Scale bars represent 200 μm. FIG. 2I is a representative cryosection image of wk5 hSCAs for atrial marker MLC-2a and ventricular marker MLC-2v. Scale bar represents 200 μm. FIG. 2J is an illustration of beating pattern of wk5 hSCAs analyzed using MEA. Heatmap shows representative pattern of propagated conduction. Error bars represent SEM.

FIGS. 3A-3G illustrate sympathetic regulation in hSCAs. FIG. 3A is a representative 3D reconstructive image of wk5 hSCAs using light sheet microscopy for cTnT and PRPH. Scale bars represent 100 μm, and 10 μm for the inset. White arrows indicate the nodal structure alone symN axons. FIG. 3B is a representative cryosection image of wk5 hSCAs for symN axonal labeling using the combinations of cTnT/VMAT2 and TH/a-actinin. White arrows indicate the nodal structure alone symN axons. Scale bars represent 200 μm. FIG. 3C is a representative TEM image showing the physical contact between symN axon (A) and heart muscles (Myo). White dashed line delineates the border of symN axonal terminal and muscle. Arrow indicates the synaptic structure. Scale bar represents 200 nm. FIG. 3D is a representative whole mount image of wk5 hSCAs using NS510. Scale bar represents 200 μm. FIG. 3E is a bar graph showing NE level in hSCA cell lysates was quantified by ELISA. n=4 biological replicates. FIG. 3F Left: is a micrograph of Ca²⁺ imaging capturing the functional coupling between symNs and cardiac tissues. Right: Quantification of the Ca²⁺ imaging recording showed the causal effect of symN activity to CM responsiveness. Scale bar represents 200 μm. FIG. 3G is bar graph showing SymNs in wk5 hSCAs were activated by nicotine (NIC) or blue light. The changes of cardiac beating were quantified using image-based hSCA beating analysis. Unpaired Student's t test. n=4 biological replicates. Error bars represent SEM. *, P<0.05.

FIGS. 4A-4D illustrate SymNs regulate cardiac development through NE signaling in hSCAs. FIG. 4A is a graph showing selected upregulated GO terms that were involved in cardiac development and regulation during symN differentiation. FIG. 4B is a schematic illustration of labetalol (LAB) treatment during hSCA development. FIG. 4C is: Left: Representative bright field image of wk5 hSCAs treated with LAB or DMSO. Right: Cell count of wk5 hSCAs treated with LAB or DMSO. Unpaired Student's t test. n=6 biological replicates. Scale bar represents 200 μm. FIG. 4D is a series of bar graphs showing the results of RT-qPCR analysis of wk5 hSCAs with LAB or DMSO for cardiac maturity markers. Unpaired Student's t test. n=4 biological replicates. Error bars represent SEM. *, P<0.05. **, P<0.01.

FIGS. 5A-5G show hSCAs model hypoxia-induced infarction. FIG. 5A is a schematic illustration of hypoxia-induced cardiac infarction model on wk5 hSCAs. FIG. 5B is Left: Representative whole mount images of wk5+10 days hSCAs labeled with Image-iT hypoxic dye. Right: Quantification of Image-iT intensity. Unpaired Student's t test. n=6 biological replicates. Scale bar represents 200 μm. FIG. 5C is NE levels of hSCAs in normoxic or hypoxic environments measured by NS510 staining (n=4 biological replicates) or ELISA (n=3 biological replicates). Unpaired Student's t test. FIG. 5D is representative TEM images showing the extracellular matrix collagen (C) that is associated with the myofibers (myo). Scale bar represents 200 nm. FIG. 5E is Left: Schematic illustration of stiffness measurement for hSCAs using AFM Right: AFM confirmed the increased stiffness of hypoxic hSCAs. Unpaired Student's t test. n=4 biological replicates. FIG. 5F is a representative cryosection image of wk5+10 days hSCAs for cell death and fibrosis markers. Scale bars represent 200 μm. FIG. 5G is a series of bar graphs showing the results of RT-qPCR analysis of wk5+10 days hSCAs for calcium handling genes. Ordinary one-way ANOVA. n=4 biological replicates. Error bars represent SEM. *, P<0.05. **, P<0.01. ****, P<0.0001.

FIG. 6 is a plot showing representative quantification of image-based hSCA beating analysis using the ImageJ Time Series Analyzer.

FIG. 7 is a plot showing force to deformation extension plot of AFM analysis of hSCAs.

### DETAILED DESCRIPTION OF THE INVENTION

### I. Definitions

The disclosed method and compositions can be understood more readily by reference to the following detailed description of particular embodiments and the Example included therein and to the Figures and their previous and following description.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of proprioceptors, or a population of undifferentiated stem cells. Alternatively, the population may comprise more than one cell type, for example a mixed cell population.

As used herein, the term “stem cell” refers to a cell with the ability to divide for indefinite periods in culture and to give rise to specialized cells. A stem cell refers to a stem cell that is from a human.

As used herein, the term “embryonic stem cell” refers to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell, similar to an embryonic stem cell, formed by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples, CI 4, C72, and the like. An induced pluripotent stem cell may be prepared from any fully (e.g., mature or adult) or partially differentiated cell using methods known in the art. For example, but not by way of limitation, an induced pluripotent stem cell may be prepared from a fibroblast, such as a human fibroblast; an epithelial cell, such as a human epithelial cell; a blood cell such as a lymphocyte or hematopoietic cell or cell precursor or myeloid cell, such as a human lymphocyte, hematopoietic cell or cell precursor or human myeloid cell; or a renal epithelial cell, such as a human renal epithelial cell. In certain non-limiting embodiments, an induced pluripotent stem cell contains one or more introduced reprogramming factor associated with producing pluripotency. In certain non-limiting embodiments a human induced pluripotent stem cell is not identical to a human embryonic pluripotent stem cell.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation. Such cells vary in their differentiation capacity, but it is usually limited to cell types in the organ of origin.

As used herein, the term “neuron” refers to a nerve cell, the principal functional units of the nervous system. A neuron consists of a cell body and its processes—an axon and one or more dendrites. Neurons transmit information to other neurons or cells by releasing neurotransmitters at synapses.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as sympathetic neurons, parasympathetic neurons, sensory neurons or a subtype thereof such as nociceptors, mechanoreceptors and/or proprioceptors, etc. As used herein, the term “directed differentiation” in reference to a stem cell typically refers to the use of small molecules, growth factor proteins, and other growth conditions to promote the transition of a stem cell from the pluripotent state into a more mature or specialized cell fate (e.g., neurons or a subtype thereof).

As used herein, the term “inducing differentiation” in reference to a cell refers to changing the default cell type (genotype and/or phenotype) to a non-default cell type (genotype and/or phenotype). Thus, “inducing differentiation in/of a stem cell” refers to inducing the stem cell (e.g., stem cell) to divide into progeny cells with characteristics that are different from the stem cell, such as genotype (e.g., change in gene expression as determined by genetic analysis such as a microarray) and/or phenotype (e.g., change in expression of a protein, such as one or more markers.

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” cells with a compound refers to placing the compound in a location that will allow it to touch the cell. The contacting may be accomplished using any suitable methods. For example, contacting can be accomplished by adding the compound to a tube of cells. Contacting may also be accomplished by adding the compound to a culture medium comprising the cells. Each of the compounds can be added to a culture medium comprising the cells as a solution (e.g., a concentrated solution). Alternatively or additionally, the compounds as well as the cells can be present in a formulated cell culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “an effective amount” or “effective amounts” refers to an amount of a molecule that is sufficient to achieve a desired effect. In some examples, the amount is effective in directing the in vitro differentiating of stem cells into a population of differentiated cells expressing one or more desired markers. In certain embodiments, the population of differentiated cells includes cells expressing one or more desired marker.

As used herein, “transformed” and “transfected” encompass the introduction of a nucleic acid (e.g., a vector) into a cell by a number of techniques known in the art.

### II. Method of Making Assembloids

Provided herein are assembloids and easy to reproduce and cost-effective methods of making the same without the need for bioengineering or special instrumentation. The modular approach allows adaptation for innervation of various organoid types, e.g., cardiac, lung, kidney, or liver, with the corresponding neurons, e.g., symNs, parasymNs, or SNs. The process of assembloid assembly is typically in free floating 3D culture, which is relatively easy to work with and reproducible.

Organoids are spatially organized 3D tissues that contain multiple cell types which self-organize through similar processes as those observed in vivo (i.e., cell-sorting and spatially restricted lineage commitment) and, thus, their progressive organization is highly reminiscent of the actual organ morphogenesis (Lancaster and Knoblich, Science 345:1247125 (2014), 10.1126/science.1247125). Assembloid refers to the assembly of more than two pre-differentiated cell types into an organoid structure^{1,2,28}. More particularly, assembloids are self-organizing 3D cellular systems that result from the integration of two or organoids or the combination of organoids with missing cell types or primary tissue explants (Kanton and Pasca, Development, 149 (20): dev201120 (2020). doi: 10.1242/dev.201120).

The assembloid strategy has recently become popular, as it supports (i) the modeling of complex organs, (ii) modeling of interactions between various organs, or (iii) modeling of different regions in the body (for example in the brain)^{1,2,28}.

The provided methods typically include combining dissociated neuron progenitor cells with dissociated tissue progenitor cells and culturing them under free floating 3D culture conditions suitable for the neuron progenitor cells and tissue progenitor cells to form one or more assembloids. Preferably, the assembloid(s) is cultured under suitable conditions and duration for the neuron progenitor cells and tissue progenitor cells to mature. Maturation can include, for example, expression of one or more markers; presence of one or more structures and/or ultrastructures; and/or one or more functionalities consistent with the corresponding mature tissue or system in vivo.

In preferred forms, the combined progenitor cells self-organize/self-assemble into assembloids. Thus, in some embodiments, the cells self-organize/self-assemble in conventional 3D culture alone, and without the need for special instrumentation. For example, bioprinting such as printing of substrates, use of hydrogels, use of organ-mimicking scaffolds is a common strategy when generating complex organ-like tissues or structures with multiple cell types. In some embodiments, the methods do not include the bioprinting such as printing of substrates, use of hydrogels, and use of organ-mimicking scaffolds. In some embodiments, the cells are cultured under agitation or movement to keep them free-floating and/or prevent adhesion to the culture dish surfaces. In some forms the culture is agitated, swirled, stirred, shaken, or otherwise put in motion to place the culture medium and cells in motion in the culture or to prevent the cells from falling to the bottom of the culture. The movement can be continuous or intermittent. In some forms, the cells are cultured using a shaker, to ensure the cells remain free-floating during the culture period.

The neuron progenitor cells and tissue progenitor cells can be combined by any suitable means. For example, non-adherent cells are typically combined and/or mixed and subsequently co-cultured in a 3D environment under suitable culture conditions to maintain or induce further growth and maturation of the cells and organoids, and assembly of the organoids into assembloids. At the time of mixing, the dissociated neuron progenitor cells and/or tissue progenitor cells can be single cells, groups or clusters of cells, spheroids, organoids, or a combination thereof, provided that during formation and maturation of the assembloid, the maturing neuron progenitor cells innervate the maturing tissue progenitor cells to form an innervated organoid. In some embodiments, the neuron progenitor cells and tissue organoids are mixed in a 1:1 ratio. In other embodiments, the cells are mixed in a ratio that resembles the in vivo composition of the tissue at that developmental stage or can be determined empirically depending on the neuron and target tissue.

An exemplary, not limiting example of making assembloids is outlined in the experiments below. Day 14 symN progenitor cells and day 7 cardiac progenitor cells were dissociated by accutase. Cells were fully mix at 1:1 ratio as 100×10³ symN progenitor cells+100×10³ cardiac progenitor cells on 96-well ultra-low attachment plates to form one organoid per well, or as 500×10³ symNblasts+500×10³ cardiac progenitors on 24-well ultra-low attachment plates for bulk organoid generation (about 5 organoids will form per well). Assembloids were then fed with hSCA medium: Neurobasal medium+CDBM base at 1:1 ratio, 100× B27, 200× L-Glutamine, 200× N2, 12.5 ng/ml GDNF, 12.5 ng/ml BDNF, 12.5 ng/ml NGF, 100 μM ascorbic acid, 100 μM dbcAMP, 0.0625 μM retinoic acid (add RA freshly every feeding), and 10 μg/ml insulin (Sigma, I-034). hSCAs were fed every 3 days. In this example, the cells were mixed and cultured in low attachment plates, in floating cultures on a shaker for up to 5 weeks. The size of the assembloids increased until week 3 and stabilized after, which was in line with decreased Ki67 expression from wk1 to wk5. The assembloids were beating spontaneously with a beating rate that increased over time. Results indicated that on wk5, hSCAs have stopped proliferating and may have entered their maturation stage.

Maturation and function can be assessed using any suitable means. Such characteristics can be determined using a range of tools and techniques such as light, immunofluorescent, and electron microscopy; protein and mRNA expression analysis, physiological assays, etc. In the experiments described below, symN progenitor cells and cardiomyocyte progenitor cells were combined to form cardiac assembliods, and found to hold a variety of characteristics typical of mature, innervated cardiac tissue. Any of these criteria can be used to assess cardiac assembloids. Alternative tests and criteria for combinations of other neurons and tissues are known in the art and can be substituted for analysis of resulting assembloids.

In some forms, the methods include forming 3D neuron progenitor cells from stem cells prior to the mixing step. The neuron progenitor cells can be, for example, sympathetic neuron (symN) progenitors, parasympathetic neuron (parasymN) progenitors, sensory neuron (SN) neuron progenitors, or a combination thereof. Early progenitor cells should be differentiated as 3D spheroids to form the neuron progenitor cells. For example, the experiments below show that SOX10⁺ neural crest cells (NCCs), the progenitors of peripheral neurons, can be made in 2D, adherent cultures, with high efficiency (about 90%). By replating the differentiated NCCs into 3D spheroids, the early sympathetic progenitor markers, PHOX2B, ASCL1, HAND2, GATA2, GATA3, are induced, representing sympathetic neuroblasts (symNblast). Neurons derived from symNblasts yield high purity (about 80%) express typical symN markers, including nicotinic receptor CHRNA3/B4, adrenergic receptor ADRA2A/B2, vesicular monoamine transporter VMAT1/2, tyrosine hydroxylase TH, and norepinephrine transporter NET, and have spontaneously firing action potentials, which can be upregulated by nicotine treatment.

Methods of making neuron progenitor cells including those of not only symN, but also parasymN, and SN, are known in the art and discussed and exemplified below, and can be adapted for use in the disclosed methods and assembloids.

Likewise, in some forms, the methods include forming tissue progenitor cells from stem cells prior to the mixing step. Tissue progenitor cells can be any organ tissue that is naturally innervated by the peripheral nervous system. Examples include, but are not limited to, heart, lung, kidney, liver, salivary glands, skin, and gastro-intestinal cells. Early progenitor cells can be differentiated in 2D or as 3D organoids to form the tissue progenitor cells. For example, in experiments below, a cardiomyocyte (CM) differentiation protocol was used to generate early cardiac progenitors, which may still have the multipotency to be differentiated into several of the cardiac lineages. hPSCs were cultured in 2D, first induced by the WNT activator CHIR99021, followed by WNT inhibition using XAV939. Cardiac progenitors expressed typical markers, including GATA4, WT1, ISLET-1, and CD56 shown by RTqPCR on day 7.

Methods of making tissue progenitor cells including cardiomyocytes, are known in the art and discussed and exemplified below, and can be adapted for use in the disclosed methods and assembloids.

As discussed herein, the disclosed system is modular and thus accommodates different combinations of neuron progenitor cells and tissue progenitor cells to form different assembloids, and the culture conditions can be adjusted accordingly.

In some embodiments any one or more of the reagents and time points to methods as exemplified in the experiments below are varied up or down by any integer value within and including 15 times more or less, or any specific range there between, of the utilized concentration/amount.

### III. Assembloid Components and Methods of Making the Same

The assembloids typically include both neurons and a non-neuron tissue. As discussed herein, they are typically formed by mixing neuron progenitor cells and (non-neuron) tissue progenitor cells. Thus, neuron and tissue progenitor cells, and methods making the same are provided.

The starting cells for preparation of neuron and/or tissue progenitor cells can be stems cells. The stem cells can be totipotent, pluripotent, or multipotent. In certain embodiments, the stem cells are human stem cells. Human stem cells include human embryonic stem cells (hESC). In some forms, the human stem cells are human pluripotent stem cells (hPSC). Examples of pluripotent and multipotent stem cells include, but are not limited to, human induced pluripotent stem cells (hiPSC), human parthenogenetic stem cells, primoridal germ cell-like pluripotent stem cells, epiblast stem cells, F-class pluripotent stem cells, somatic stem cells, cancer stem cells, or any other cell capable of lineage specific differentiation. In certain embodiments, the human stem cell is a human embryonic stem cell (hESC). In certain embodiments, the human stem cell is a human induced pluripotent stem cell (hiPSC). In certain embodiments, the stem cells are non-human stem cells. Non-limiting examples of non-human stem cells include non-human primate stem cells, rodent stem cells, dog stem cells, cat stem cells, horse stem cells, pig stem cells, etc. In certain embodiments, the stem cells are pluripotent stem cells. In certain embodiments, the stem cells are embryonic stem cells. In certain embodiments, the stem cells are induced pluripotent stem cells.

The cells can be autologous, e.g. derived from the subject, or syngenic. Allogeneic cells can also be isolated from antigenically matched, genetically unrelated donors (identified through a national registry), or by using cells obtained or derived from a genetically related sibling or parent.

The starting cells for parasymNs can be Schwann or Schwann-like cells, such as induced Schwann cell progenitor cells. In some embodiments the starting cell type is stem cells that proceed to parasympathetic neurons (parasymNs) through an intermediate state in which the cells are Schwann or Schwann-like cells, preferably Schwann cell progenitor cells.

Schwann cells (SCs) are a type of glial cell that surrounds neurons, keeping them alive and sometimes covering them with a myelin sheath, and are the major glial cell type in the peripheral nervous system. They play important roles in the development, maintenance, function, and regeneration of peripheral nerves.

Neural crest cells (NCCs) are SOX10⁺ early embryonic progenitor cells that are highly migratory and give rise to a large variety of cell types, including all peripheral nervous system cells, melanocytes, and peripheral glia. It has been previously established an efficient and defined NC differentiation protocol that was shown to give rise to sensory enteric, and symNs. These NCCs can be used to derive Schwann cells (SCs), that showed identities that are similar to primary SCs and were capable of myelination (Majd, H., et al., bioRxiv 2022.08.16.504209; doi.org/10.1101/2022.08.16.504209 (2022); Cell Stem Cell, 30 (5): 632-647.e10, (2023); doi: 10.1016/j.stem.2023.04.006).

In some embodiments, the starting cell type or an intermediate cell type proceeding from a stem cell or neural crest cell starting cell type, is Schwann cell progenitor (SCP) cells.

### A. Neuron Progenitor Cells

The disclosed assembloids include neuron progenitor cells. Such neuron progenitor cells include, but are not limited to, progenitors of sympathetic (symNs), parasympathetic (parasymNs), and/or sensory neurons (SNs). The neuron progenitor cells can be prepared using any suitable means. Thus, the methods can include in vitro differentiation of cells into symN, parasymN, and/or SN progenitors.

### 1. Preparation of Sympathetic and Parasympathetic Neuron Progenitor Cells

Methods of making symN and parasymN are known in the art and non-limiting examples are provided herein. For example, efficient ways to differentiate symNs are described in Wu, H. F. & Zeltner, N. J Vis Exp (2020), Wu, et al., Nat Commun 13, 7032 (2022) doi.org/10.1038/s41467-022-34811-7), which are specifically incorporated by reference herein in their entiries. Alternatives protocols are reviewed and compared in Wu, et al., “Overview of Methods to Differentiate Sympathetic Neurons from Human Pluripotent Stem Cells,” Current Protocols Stem Cell Biology. 50 (1), e92 (2019)), and see also Frith, et al., Curr Protoc Stem Cell Biol 49, e81 (2019); Kirino, et al., Sci Rep 8, 12865 (2018)), all of which are specifically incorporated by reference in their entireties. Methods of making parasymNs were described in, for example, Wu, et al. “Parasympathetic Neurons Derived From Human Pluripotent Stem Cells Model Human Diseases and Development.” ssrn.com/abstract=4318816 dx.doi.org/10.2139/ssrn.4318816, Takayama, et al. Sci Rep 10, 9464 (2020)), and Goldsteen, Front Pharmacol. 2022; 13:991072, doi: 10.3389/fphar.2022.991072, all of which are specifically incorporated by reference in their entireties. Methods of making sensory neurons (SN) are also described. See, for example, Saito-Diaz, et al., Stem Cell Reports. 2021 Mar. 9; 16 (3): 446-457. doi: 10.1016/j.stemcr.2021.01.001, Saito-Diaz, et al., STAR Protoc. 2022 Mar. 18; 3 (2): 101187. doi: 10.1016/j.xpro.2022.101187. eCollection 2022 Jun. 17, Saito-Diaz, et al., Front. Cell Dev. Biol., 3 May 2023, Sec. Stem Cell Research, Volume 11-2023|doi.org/10.3389/fcell.2023.1101423, Deng, et al., Stem Cell Reports. 2023 Apr. 11; 18 (4): 1030-1047, and Young, et al., Mol Ther. 2014 August; 22 (8): 1530-1543, doi: 10.1038/mt.2014.86, all of which are specifically incorporated by reference herein.

The direct comparison of various symNs is reviewed in Wu, et al., “Overview of Methods to Differentiate Sympathetic Neurons from Human Pluripotent Stem Cells,” Current Protocols Stem Cell Biology. 50 (1), e92 (2019), specifically incorporated herein in its entirety. For example, a differentiation protocol for the generation of autonomic neurons with symN character is described in Zeltner, N. et al. “Capturing the biology of disease severity in a PSC-based model of familial dysautonomia.” Nature Medicine. 22 (12), 1421-1427 (2016), specifically incorporated herein in its entirety. This protocol uses KSR-based medium, which was used in both the maintenance of undifferentiated hPSCs and cell differentiation. Furthermore, hPSCs were maintained on mouse embryonic fibroblasts (MEF feeder cells). This protocol and PSCs from patients with FD to model the disorder (Zeltner, N. et al. “Capturing the biology of disease severity in a PSC-based model of familial dysautonomia.” Nature Medicine. 22 (12), 1421-1427 (2016), which is specifically incorporated by reference herein in its entirety), and a more detailed version was described in Saito-Diaz, et al., “Autonomic Neurons with Sympathetic Character Derived From Human Pluripotent Stem Cells. Current Protocols Stem Cell Biology,” 49 (1), e78 (2019), which is specifically incorporated by reference herein in its entirety. The neural fate was induced by dual SMAD inhibition to block TGF-β and BMP signaling in the first 2 days. WNT activation using CHIR99021 promoted neural progenitors to become NC cells. On day 11, cells were sorted by FACS for CD49D+ or SOX10+ populations which yielded about 40% NC generation efficiency. Thus, sorting was used to ensure the efficiency and purity for the next steps of differentiation. The NCCs were maintained and amplified as spheroids with the combined treatment of FGF2 and CHIR. After 4 days, the NC spheroids of maintenance were plated and given BDNF, GDNF, and NGF to finish the symN maturation. Although these symNs expressed strong symN markers such as ASCL1, TH, DBH, and PHOX2A, markers for more mature symNs, including expression of the nicotinic acetylcholine receptor (CHRNA3/CHRNB4) and vesicle transporter (VMAT1/2), were low even after 70 days of differentiation. HOX genes in this protocol were not formally tested, and mature neural properties, including electrophysiological activity of the cells, were not verified.

An improved protocol is described in Wu and Zeltner, “Efficient Differentiation of Postganglionic Sympathetic Neurons using Human Pluripotent Stem Cells under Feeder-free and Chemically Defined Culture Conditions,” Published: May 24, 2020 doi: 10.3791/60843, which is specifically incorporate by reference herein in its entirety.

Briefly, HPSCs are maintained in feeder-free conditions, on vitronectin (VTN)-coated dishes, using Essential 8 (E8) media. The formula of the differentiation media is modified at each stage, thereby increasing the percentage of the NC population. To be more specific, at early stage, TGF-β inhibition that stimulate neuroectoderm cell fate is supported with Wnt activator and precise level of BMP4, which mimics the environment of neural tube formation. Subsequent treatment of Wnt activator mimics NC induction. This protocol results in significantly improved NC efficiency, which is about 80%. The differentiated NCs are further primed in 3D spheroid culture to form symNblast. This spheroid stage turns out to be highly selective, meaning only CD49D+/SOX10+ NCs can survive and form spheroids, while the 20% misdifferentiated cells will be eliminated. Therefore, the symN maturation can be done on CD49D+/SOX10+ sorted or unsorted bulk NCC populations. Both show high levels of symN marker expression by day 30. In addition, day 10 NCs produced from the protocol can be cryopreserved. These improvements make the protocol highly efficient, accessible, and allow high-scale NC and symN production. Moreover, the symNs generated with this protocol are responsive to electrophysiological, and recording and to treatments with symN activator and inhibitor compounds. SymN prepared in this manner are utilized in the experiments below.

Culture steps, differentiation factors, and methods of in vitro differentiation cells into parasympathetic neurons (parasymNs) are provided. Any of the methods can include one or more of (1) inducing stem cells (e.g., ESCs or IPSCs) to differentiate into neural crest cells (NC), and in the case of parasymN, (2) inducing NCs to differentiate into SCPs.

a. NC differentiation

Prior to differentiation, plates are typically coated with a substrate such as Geltrex or vitronectin, and used to culture stem cells, followed by induction to differentiate into neural crest cells (NC cells or NCC).

Cells are fed and cultured over time and may or may not include one, two, three, or more replatings. In some embodiments, the cells are stabilized, e.g., after thawing, e.g., by splitting 2-3× after thawing. The cells can be cultured in a monolayer, neurospheres, or a combination thereof. In some embodiments, a feeder layer is utilized, e.g., mouse fibroblasts. In some embodiments, the methods are also free from a feeder layer.

The disclosed methods and compositions for preparing NC cells can include one or more compositions or methods or steps thereof disclosed in U.S. Patent Application Nos. 2019/0093074 and 2022/0195386, U.S. Pat. No. 9,453,198, Wu, H. F., Zeltner, et al., “Efficient Differentiation of Postganglionic Sympathetic Neurons using Human Pluripotent Stem Cells under Feeder-free and Chemically Defined Culture Conditions,” J. Vis. Exp. (159), e60843, doi: 10.3791/60843 (2020), each if which is specifically incorporated by reference herein in its entirety.

In some embodiments, stem cells are plated on basement membrane matrix at day 0, feed with differentiation media for about 10 days (e.g., about 8, 9, 10, 11, or 12 days) beginning on day 0 or day 1. In some embodiments, two differentiation medias are used, a first differentiation media for about 1 day, followed by a second differentiation media for the remainder of the NC differentiation period.

The methods can include transforming growth factor beta (TGFB)/Activin-Nodal signaling inhibition and/or wingless (Wnt) signaling activation and/or inhibition of Rho-binding kinase (i.e., ROCK inhibition). The methods can include BMP supplementation, e.g., BMP4. The methods thus can include culturing or otherwise contacting the cells with one or more TGFβ/Activin-Nodal signaling inhibitors and/or one or more Wnt signaling activators and/or one or more ROCK inhibitors and/or one or more BMPs. Typically the inhibitor(s) and/or activator(s) and/or BMP(s) are used in a suitable combination and effective amounts and for sufficient duration to differentiate stems cells, and/or induce them to form and/or maintain them as NC cells, e.g., as exemplified herein.

Non-limiting examples of inhibitors of TGFβ/Activin-Nodal signaling are disclosed in WO2011/149762, and are otherwise known in the art. In certain embodiments, the inhibitor of TGFβ/Activin-Nodal signaling is a small molecule selected from the group consisting of SB431542, derivatives thereof, and mixtures thereof. “SB431542” refers to a molecule with a number CAS 301836-41-9, a molecular formula of C₂₂K₈N₄O₃, and a name of 4-[4-(1,3-benzodioxol-5-yl)-5-(2-pyridinyl)-1H-imidazol-2-yl]-benzamide. In some embodiments, the inhibitor of TGFβ/Activin-Nodal signaling, e.g., SB431542, is used in a concentration ranging from 5 μM to 10 μM inclusive. A particularly preferred concentration is about 10 μM, or 10 μM. In some embodiments when two differentiation medias are used, this molarity can be used in both a first differentiation media and a second differentiation.

The ROCK inhibitor can be any inhibitor as long as it inhibits the function of Rho-binding kinase. Examples of the ROCK inhibitor include GSK269962A (Axon medchem), Fasudil hydrochloride (Tocris Bioscience), Y-27632 and H-1152 (all from, Wako Pure Chemical). A preferred example includes Y-27632. In some embodiments, the ROCK inhibitor, e.g., Y-27632, is used in a concentration ranging from 5 μM to 10 μM inclusive. A particularly preferred concentration is about 10 μM, or 10 μM. In some embodiments when two differentiation medias are used, about 10 μM, or 10 μM, of Y-27632 molarity is used in the first differentiation media and absent in second differentiation.

Non-limiting examples of bone morphogenic proteins (BMPs) include BMP2, BMP4, BMP6, and BMP7. In some embodiments, the BMP, e.g., BMP4, is used in a concentration ranging from 0 ng/ml to 5 ng/ml inclusive. A particularly preferred concentration is about 1 ng/ml, or 1 ng/ml. BMP, e.g., BMP4 can be important, and is preferably used at the lower end of a range that gives the desired results, e.g., as discussed herein such as efficiently obtaining NC. Thus, in some embodiments the BMP, e.g., BMP4 is titrated, particularly down, to determine the best concentration, which may vary somewhat between different cell types, e.g., different hPSC lines. In some embodiments, when two differentiation medias are used, BMP is present in the first differentiation media and absent from the second differentiation media.

In certain embodiments, the activator of Wnt signaling lowers GSK3β for activation of Wnt signaling. Thus, the activator of Wnt signaling can be a GSK3β inhibitor. Non-limiting examples of activators of Wnt signaling or GSK3β inhibitors are disclosed in WO2011/149762, and Calder et al., J Neurosci. 2015 Aug. 19; 35 (33): 11462-81, which are incorporated by reference in their entireties, and are otherwise known in the art. In certain embodiments, the activator of Wnt signaling is a small molecule selected from the group consisting of CHIR99021, derivatives thereof, and mixtures thereof “CHIR99021” (also known as “aminopyrimidine” or “3-[3-(2-Carboxyethyl)-4-methylpyrrol-2-methylidenyl]-2-indolinone”) refers to IUPAC name 6-(2-(4-(2,4-dichlorophenyl)-5-(4-methyl-1H-imidazol-2-yl)pyrimidin-2-yla-mino) ethylamino)nicotinonitrile. In some embodiments, the activator of Wnt signaling, e.g., CHIR99021, is used in a concentration ranging from 100 nM to 500 nM from days 0-2 and/or 0.25 μM to 1.5 μM from day 2 on, inclusive. In embodiments, when two differentiation medias are used, the activator of Wnt signaling, e.g., CHIR99021, is used at a concentration of 300 nM in the first differentiation media, and/or 0.75 μM CHIR99021 in the second differentiation media.

For example, on day 0, hPSCs were replated on Geltrex (Invitrogen, A1413202)-coated plates at 125×10³ cells/cm². Day 0-1 a first differentiation medium composed of Essential 6 medium supplemented with 0.4 ng/ml BMP4, 10 μM SB431542 and 300 nM CHIR99021. From day 2 on, cells were fed with a second differentiation medium composed of Essential 6 medium supplemented with 10 μM SB431542 and 0.75 μM CHIR99021.

In another example, NCC differentiation is initiated (D0) by aspirating the maintenance medium (E8) and replacing it with a first neural crest induction medium [BMP4 (1 ng ml-1), SB431542 (10 μM), and CHIR 99021 (600 nM) in Essential 6 medium]. Subsequently, on D2 a second neural crest induction medium [SB431542 (10 μM) and CHIR 99021 (1.5 μM) in Essential 6 medium] was fed to the cultures until D12.

NC stage (e.g., days 10-12) cells can be frozen and stored for later use. In some embodiments, the CryoPause method (Wong, et al., Stem Cell Reports 9, 355-365 (2017)) is utilized.

b. Schwann Cell Progenitors

In some embodiments, particularly those including parsymN, the methods include inducing a starting cell type to form Schwann Cell Progenitor (SCP) cells.

For example, beginning with NCC, including but not limited to those formed according to the methods described above, Schwann cells can be induced according methods that are known in the art including, but not limited to, Majd, et al., “Deriving Schwann Cells from hPSCs Enables Disease Modeling and Drug Discovery for Diabetic Peripheral Neuropathy,” Cell Stem Cell, 2023 May 4; 30 (5): 632-647.e10. doi: 10.1016/j.stem.2023.04.006., and Kim, et al., “Schwann Cell Precursors from Human Pluripotent Stem Cells as a Potential Therapeutic Target for Myelin Repair”, Stem Cell Reports 8, 1714-1726 (2017), doi: 10.1016/j.stemcr.2017.04.011, each of which is specifically incorporated by reference herein in its entirety.

SCs are thought to arise from SOX10+ NC cells in a stepwise process. For example, based on studies in the mouse and chick embryos, NC first gives rise to SC precursors that are competent to associate with neuronal fiber bundles in the developing nerves. The associated neurons produce NRG1 which promotes the survival and further differentiation of SC precursors (SCPs) by activating ERBB3 receptors. By E13.5 of mouse development, SC precursors give rise to immature SCs which express lineage-specific markers such as GFAP, S100 and POU3F1 while maintaining the expression of SOX10. Terminal differentiation of SCs into myelinating and non-myelinating fates continues for extended time periods and concludes only after birth.

In some embodiments, NCC, including but not limited to day 10-12 NCC prepared according to the methods discussed above, are replated as spheroids until about day 24 during which the cells are induced to form Schwann Cell Progenitors. For example, in some embodiments, NC monolayers are detached, e.g., using accutase. Cells are collected and replated as 3D spheroids, and cultured in medium supplemented with L-Glutamine, N2 and B27, an activator of Wnt signaling, e.g., CHIR99021, FGF2, and NRG1. In a particular embodiment, the media is Neurobasal media containing with L-Glutamine, FGF2 (10 ng ml-1), CHIR 99021 (3 μM), N2 supplement (10 μl ml-1), B27 supplement (20 μl ml-1), and NRG1 (10 ng/ml). Preferably, the spheroids are cultured on ultra-low-attachment plates to form free-floating 3D developing precursors. Such media can be referred to SC precursor media (SCP).

In other embodiments, colonized hPSCs can be re-plated onto growth factor-reduced Matrigel-coated culture dishes. The next day, the culture medium was switched to culture medium containing N2, B27, BSA, GlutaMAX, β-mercaptoethanol, CT 99021, and SB431542. In a specific embodiment, the media is advanced DMEM/F12 and Neurobasal medium (1:1 mix) supplemented with 1× N2, 1× B27, 0.005% BSA, 2 mM GlutaMAX, 0.11 mM β-mercaptoethanol, 3 μM CT 99021, and 20 μM SB431542. After about 6 days of differentiation, the medium is further supplemented with NRG1 (e.g., 50 ng/mL). In some embodiments, media is changed daily. These Schwann Cell Progenitors can be routinely dissociated with Accutase treatment upon reaching 80% confluence and were expanded by additional cultivation in the foregoing differentiation media. The hPSC-derived SCPs can be generated after approximately 18 days of differentiation. The foregoing media can be used for the induction and maintenance of hPSC-derived SCPs.

As a non-limiting example, day 10 NCCs were replated as spheroids in SCP medium until day 24. Spheroids were plated on PO/LM/FN coated plates without dissociation in SC differentiation medium that contains Neurobasal medium, B27, L-Glutamine, 10 ng/ml FGF2, 100 μM dbcAMP, and 20 ng/ml NRG1. SC identity was evaluated on day 30.

c. Parasympathetic Neuron (parasymNs) Differentiation

Exemplary compositions and methods for inducing parasymNs are discussed in publication supra and provided herein. The disclosed methods and compositions can include one or more compositions or methods or steps thereof disclosed in U.S. Ser. No. 18/503,100.

The parasymNs are typically derived from SCPs. Optionally, but preferably, the SCPs are prepared from NCC. Optionally, but preferably, the NCC are prepared from stem cells.

In preferred embodiments, the SCPs are multipotent, but not yet determined to the SC fate. Preferably, the cells have relatively high levels of SCP markers (such as PAX3/GAP43)) and relatively lower levels of immature SC makers (e.g., iSC, CD56), and differentiated SC markers (e.g., MPZ, S100β), e.g., compared to SCs. Results show that SCP genes decreased from day 16-20, accompanied by increasing iSC marker, which dropped after day 24, while SC genes were still increasing. These results demonstrate a clear progress of SC differentiation and indicate that the proper timing to start parasymN induction can be between day 6-14 after plating spheroids (e.g., 16-24 days after initiation of NCC induction). Further experiments indicated that replating before day 6 (about day 6 from NCC induction, when SCP induction is initiated at day 10) induced neuron-like cell morphology, and the appearance of well-developed neurite bundles. Thus, in some embodiments, ParasymNs induction is initiated in SCPs about 4-14 days after plating NCC spheroids (i.e., 14-26 days after inducing NCC differentiation from stem cells).

ParasymNs are typically induced using a combination of one or more of Glial Cell Derived Neurotrophic Factor (GDNF), Brain-derived Neurotrophic Factor (BDNF), Ciliary Neurotrophic Factor (CNTF), Fetal Bovine Serum (FBS), and Nerve Growth Factor (NGF). Preferably the media includes all of GDNF, BDNF, CNTF, and FBS. NGF is believed to be important for symN survival, but results show parasymN can be induced without it. Thus, in some embodiments, NGF is excluded from the media. This may reduce potential symN contamination from NCCs that are not fully differentiated. In some embodiments, the neural supplement BrainFast is added. For example, SCP spheroids can be plated on e.g., PO/LM/FN-coated plates and cultured in media containing B27, L-Glutamine, FBS, GDNF, BDNF, CNTF, ascorbic acid, dbcAMP, and retinoic acid. In a more particular embodiments, the media is Neurobasal medium with B27, L-Glutamine, 1% FBS, 25 ng/ml GDNF, 25 ng/ml BDNF, 25 ng/ml CNTF, 200 μM ascorbic acid, 0.2 mM dbcAMP, and 0.125 μM retinoic acid optionally, but preferably, added freshly every feeding.

ParasymNs can be differentiated over about two weeks and can be maintained in parasymN differentiation medium by halfway feeding weekly.

In some embodiments, parasymNs are characterized by one or more of a neuron-like cell morphology, the appearance of well-developed neurite bundles, expression of one or more autonomic markers such as ASCL1, PHOX2B, PRPH, and/or CHRNA3, one or more parasympathetic markers such as ChAT, VAChT, ChT, and/or NPY2R, reduced expression of SOX10 e.g., relative to SCPs and/or SCs, predominate expression of ChAT relative TH, e.g., compared to symNs, HOX 1-5 positive, optionally except HOX 2 (e.g., HOX 1-5⁺, but HOX²⁻, also referred to HOX 1⁺, 3⁺, 4⁺,5⁺, HOX²⁻), one or more cholinergic muscarinic receptors (MusR), preferably at least M2 and/or M4, functionally activated by bethanechol (BeCh), functionally inactivated by atropine, insensitivity to 6-OHDA, ability to build functional connectivity to and/or innervate target tissues e.g., cardiomyocytes (CMs) (e.g., by forming neurocardiac junctions, optionally nodal structures that are sensitive to nicotine), adipocytes, etc. and/or other biochemical, morphological, and/or functional features discussed in more detail in the experiments below. Markers can be measured quantitatively or qualitatively in any suitable way, for example, gene expression can be assessed by RT-PCR, protein expression can be assessed by Western Blot, immunofluorescence, etc.

In some embodiments, the methods are carried out under conditions that yield about 50% or more ChAT⁺ parasymNs optionally but preferably in combination with very high differentiation specificity as indicated by almost all (e.g., at least 70%, 75%, 80%, 85%, 90%, 95%, or more) PRPH⁺ neurons expressing ChAT.

In a specific embodiment, parasymNs are differentiated from SCPs. Day 10 NCCs were dissociated using accutase and aggregated to make spheroids on ultra-low attachment plates in SCP medium containing Neurobasal medium, B27, L-Glutamine, 3 μM CHIR99021, 10 ng/ml FGF2, and 10 ng/ml NRG1. On day 16, SCP spheroids are dissociated using accutase and replated on PO/LM/FN-coated plates at 100×10³ cells/cm² in parasymN differentiation medium is Neurobasal medium including B27, L-Glutamine, 1% FBS, 25 ng/ml GDNF, 25 ng/ml BDNF, 25 ng/ml CNTF, 200 μM ascorbic acid, 0.2 mM dbcAMP and 0.125 μM retinoic acid (add freshly every feeding).

In some forms, dissociated day 16 SCPs (similar to day 14 symNblasts) are used to make assembloids. They can be mixed with progenitors of desired organs in the ratios and timepoints specified above.

Typically the method yields functional parasymNs. Function can be measured using any suitable method.

d. Sympathetic Neuron (symNs) Differentiation

Methods for inducing symNs are discussed in the publications supra and exemplified in the experiments below. In some embodiments, Day 10 NCCs are dissociate, e.g., using accutase, and aggregated to generate spheroids, e.g., on ultra-low attachment plates in medium. The medium typically contains an effective amount of an activator of Wnt signaling, e.g., CHIR99021, used in a concentration ranging from 0.3-3 μM. In some embodiments, the amount of CHIR99021 is exactly 0.3 μM from day 0-2 and 0.75 μM from day 2-10 and 3 μM from day 10-14. In a particular example, the media also contains Neurobasal medium, B27, L-Glutamine, and/or FGF2, e.g., Neurobasal medium, B27, L-Glutamine, 3 μM CHIR99021 and 10 ng/ml FGF2. Minimal spheroids can be prepared in this way in about 5 days (i.e., days 10-14). Spheroids can be optionally expanded by feeding with medium containing retinoic acid in a concentration ranging from 0.05 μM-0.2 μM, where about 0.125 μM or 0.125 μM is preferred, for up to about 14 days or more.

In a specific embodiment day 10 NCCs are dissociated by accutase (Coring, AT104500) and replated to ultra-low attachment plates to form symNblast spheroids. The symNblast medium for days 10-14 contains Neurobasal medium (Gibco, 21103-049), B27 (Gibco, 17502-048), N2 supplement (Thermo Fisher/Gibco, 17502048). L-Glutamine (Thermo Fisher/Gibco, 25030-081), 3 μM CHIR99021 and 10 ng/ml FGF2 (R&D Systems, 233-FB/CF).

The cells are typically used for assembloid formation at a progenitor stage, e.g., day 10 and 14 for symNBlasts, as illustrated below. Other neuron progenitor cells can also be selected for use in assembloid formation at a similar or equivalent precursor state of differentiation.

Progenitor cells can be further differentiated, as discuss in more detail below, for more that 100 days into mature symNs, but these mature cells may or may not incorporate into an organoid. Thus, progenitor cells are typically preferred for both symNs and other neuronal cell types.

In some embodiments, following preparation of minimal or expanded spheroids, the spheroids can be dissociated using accutase and replated e.g., on PO/LM/FN coated plates in symN differentiation medium. Such media can contain retinoic acid in a concentration ranging from 0.05 μM-0.2 μM. In some embodiments, the amount of retinoic acid is about 0.125 μM or 0.125 μM, preferably added freshly at every feeding. In some embodiments, the media also contains Neurobasal medium, B27, L-Glutamine, GDNF, BDNF, NGF, ascorbic acid, and/or dbcAMP, e.g., Neurobasal medium, B27, L-Glutamine, 25 ng/ml GDNF, 25 ng/ml BDNF, 25 ng/ml NGF, 200 μM ascorbic acid, 0.2 mM dbcAMP and 0.125 μM retinoic acid. In some embodiments, Y27632, e.g., 10 μM Y27632 is added, optionally only on day 14.

Typically the method yields functional symNs. Function can be measured using any suitable method.

### 2. Preparation of Sensory Neurons

Prior to differentiation, plates are typically coated with a substrate such as Geltrex or vitronectin or Matrigel, and used to culture stem cells, followed by induction to differentiate into sensory neurons. Cells are fed and cultured over time and may include one, two, three, or more, but preferably only one replating. The cells can be cultured in a monolayer. Thus in some embodiments, the methods do not include forming neurospheres. In some embodiments, the methods are also free from a feeder layer.

It has been discovered that (i) initiating the differentiation the day of stem cell seeding (e.g., rather than 24 h later), (ii) lowering the concentration of Wnt signaling activator (e.g., CHIR99021) and, (iii) using vitronectin (VTN, rather than Geltrex) resulted in dense NC ‘ridges’. Thus, in some embodiments, the disclosed methods include (i), (ii), (iii), or a combination thereof.

The disclosed methods and compositions can include one or more compositions or methods or steps thereof disclosed in U.S. Patent Application No. 2019/0093074, U.S. Pat. No. 9,453,198, U.S. Published Application No. 2022/0195386-A1, or Saito-Diaz, et al., “Derivation of Peripheral Nociceptive, Mechanoreceptive, and Proprioceptive Sensory Neurons from the same Culture of Human Pluripotent Stem Cells,” Stem Cell Reports. 2021 Mar. 9; 16 (3): 446-457. doi: 10.1016/j.stemcr.2021.01.001, each of which is specifically incorporated by reference herein in its entirety.

The methods can include FGFR/VEGFR inhibition (e.g., SU-5402) and/or Notch inhibition (e.g., DAPT) and/or transforming growth factor beta (TGFβ)/Activin-Nodal signaling inhibition and/or wingless (Wnt) signaling activation. The methods can include BMP supplementation, e.g., BMP4. The methods thus can include culturing or otherwise contacting the cells with one or more FGFR/VEGFR and/or Notch and/or TGFβ/Activin-Nodal signaling inhibitors, and/or one or more Wnt signaling activators and/or one or more BMPs. Typically the inhibitor(s) and/or activator(s) and/or BMP(s) are used in a suitable combination and effective amounts and for sufficient duration to differentiate stems cells, and/or induce them to form and/or maintain them as sensory neurons, or a particular subpopulation thereof, e.g., as exemplified herein.

Non-limiting examples of inhibitors of FGFR family signaling are disclosed in WO2011/149762 which is incorporated by reference in its entirety, and are otherwise known in the art. In certain embodiments, the one or more inhibitor of FGF receptor family signaling is a small molecule selected from the group consisting of SU5402, PD-161570, PD-173074, derivatives thereof, and mixtures thereof. In certain embodiments, the one or more inhibitor of FGF receptor family signaling is SU5402. “SU5402” and “SU-5402” refers to a small molecule with a chemical formula of C₁₁H₁₆N₂O₃ and chemical name: 2-[(1,2-Dihydro-2-oxo-3H-indol-3-ylidene) methyl]-4-methyl-1H-pyrrole-3-pr-opanoic acid. In some embodiments, the inhibitor of FGFR family signaling, e.g., SU5402, is used in a concentration ranging from 1 μM to 5 μM inclusive. A particularly preferred concentration is about 2.5 μM, or 2.5 μM.

In certain embodiments, the inhibitor of Notch signaling is a γ-secretase inhibitor. γ-secretase inhibitors are a class of agents that prevent the generation of the active domain of Notch molecules resulting in suppression of downstream Notch signaling. Non-limiting examples of γ-secretase inhibitors are DAPT, a tripeptide aldehyde inhibitor, a γ-secretase inhibitor XII, LY-411,575. In certain embodiments, the one or more inhibitor of Notch signaling is a small molecule selected from the group consisting of DAPT, a tripeptide aldehyde inhibitor, a γ-secretase inhibitor XII, LY-411,575, derivatives thereof, and mixtures thereof. In some embodiments, the inhibitor of Notch signaling, e.g., DAPT, is used in a concentration ranging from 1 μM to 5 μM inclusive. A particularly preferred concentration is about 2.5 μM, or 2.5 μM.

Non-limiting examples of inhibitors of TGFβ/Activin-Nodal signaling are disclosed in WO2011/149762, and are otherwise known in the art. In certain embodiments, the inhibitor of TGFβ/Activin-Nodal signaling is a small molecule selected from the group consisting of SB431542, derivatives thereof, and mixtures thereof. “SB431542” refers to a molecule with a number CAS 301836-41-9, a molecular formula of C₂₂K₈N₄O₃, and a name of 4-[4-(1,3-benzodioxol-5-yl)-5-(2-pyridinyl)-1H-imidazol-2-yl]-benzamide. In some embodiments, the inhibitor of TGFβ/Activin-Nodal signaling, e.g., SB431542, is used in a concentration ranging from 5 μM to 10 μM inclusive. A particularly preferred concentration is about 10 μM, or 10 μM.

Non-limiting examples of bone morphogenic proteins (BMPs) include BMP2, BMP4, BMP6, and BMP7. In some embodiments, the BMP, e.g., BMP4, is used in a concentration ranging from 0 ng/ml to 5 ng/ml inclusive. A particularly preferred concentration is about 1 ng/ml, or 1 ng/ml. BMP, e.g., BMP4 can be important, and is preferably used at the lower end of a range that gives the desired results, e.g., as discussed herein such as efficiently obtaining NC. Thus, in some embodiments the BMP, e.g., BMP4 is titrated, particularly down, to determine the best concentration, which may vary somewhat between different cell types, e.g., different hPSC lines.

Activators of Wnt signaling are introduced above and the same compounds can be used for sensory neuron induction. In some embodiments, the activator of Wnt signaling, e.g., CHIR99021, is used in a concentration ranging from 100 nM to 500 nM from days 0-2 and/or 0.25 μM to 1.5 μM from day 2 on, inclusive. In particular embodiments the activator of Wnt signaling, e.g., CHIR99021, is used at a concentration of 300 nM from day 0-2 and/or 0.75 μM from day 2 on.

In some embodiments, the concentration of Wnt signaling activator, e.g., CHIR99021, is used at a lower level than other methods of making sensory neurons. In particularly embodiments the concentration of Wnt signaling activator, e.g., CHIR99021, is lower than a method of making sensor neurons that fails make mechanoreceptors, and optionally fails to make nociceptors, mechanoreceptors, and/or proprioceptors are in ratios similar to those found in the dorsal root ganglia.

For example, results show that prior to differentiation, when plates can be coated with Geltrex at 1:100 dilution and stored at 4° C. overnight, or vitronectin. The next day, hPSCs were harvested as using EDTA for 15 min and plated at a density of 200,000 cells/cm2. On days 0 (day of plating) to 1 of the differentiation, cultures were fed with Essential 6 Medium containing 10 UM of TGFβ/Activin-Nodal signaling inhibitor SB431542, 1 ng/mL BMP4, 300 nM Wnt signaling activator CHIR99021, and 10 μM Y-27632. On day 2, NCC D2+ media was made with Essential 6 Medium containing 10 μM SB431542 and 1.5 μM or 0.75 μM CHIR99021. For SN induction, cells were maintained in D2-12 media containing 10 μM SB431542, 0.75 μM CHIR99021, 2.5 μM of FGFR inhibitor SU5402 and 2.5 μM of Notch inhibitor DAPT. Cells were fed every 48 h between day 2 and 12.

The methods typically include a step of replating. Neural crest cells (NCCs) can be pushed towards SNs by replating them on day 12 in a combination of SN-favoring growth factors (Chambers, et al., Nat. Biotechnol. 30, 715-720 (2012), which is specifically incorporated by reference herein in its entirety), with the addition of DAPT (day 12-20), which reduces the number of SOX10+ progenitor cells and enriches the purity of the culture. Thus, in some embodiments, replating is on culture days 10, 11, 12, 13, 14 or 15, optionally in combination with SN-favoring growth factors. In the experiments below, on day 12, sensory neuron cultures were replated at a density of 250,000 cells/cm² onto plates coated with 15 μg/ml poly-L-ornithine hydrobromide and 2 μg/ml mouse-laminin-1, and 2 μg/ml human fibronectin (PO/LM/FN). Cells were dissociated with Accutase for 20 min, washed with PBS and resuspended in Sensory Neuron Media: Neurobasal media containing N2 supplement, B-27 supplement, 2 mM L-glutamine, 20 ng/ml each of GDNF, BDNF, 25 ng/ml NGF, 600 ng/ml of laminin-1, 600 ng/ml fibronectin, 1 μM DAPT and 0.125 μM retinoic acid. 20 ng/ml NT-3 was added where indicated. Cells were fed every 2-3 days through D20. On day 20, DAPT was removed and cells were fed every 3-4 days.

It was also found that SNs can be replated anytime between day 16 and day 50 and form a dense network 20 days post-replating. The experiments below, this late-replating of sensory neuron cultures was onto plates coated with 15 μg/ml poly-L-ornithine hydrobromide, 2 μg/ml of mouse-laminin-1 and 2 μg/ml human fibronectin. To replate, cells were dissociated with Accutase (Innovative Cell Technologies Inc., 84 #AT-104) for 45 min, washed once with PBS and resuspended in Sensory Neuron Media Cells with a p1000. SNs were then seeded onto the new plates at a density of 250,000 cells/cm2. Cells were fed every 2-3 days through day 20 or every 3-4 days afterwards.

Sequential culture of hESCs resulted in decreased NCC and SN differentiation efficiency over time, and thus is avoided in some embodiments.

Additionally or alternatively, this limitation could be overcome using the CryoPause method (Wong, et al., Stem Cell Reports 9, 355-365 (2017)).

It was also discovered that the cells could be frozen at the NCC stage (e.g., day 12).

In some embodiments, the method utilizes chemically defined conditions in monolayer culture to generate a combination of nociceptors, mechanoreceptors, proprioceptors.

In some embodiments, the method generates SN cells out of 60-70% of the cells.

In some embodiments, nociceptors, mechanoreceptors, and/or proprioceptors are in ratios similar to those found in the dorsal root ganglia. In some embodiments, the starting stem cells are ESC or iPSC.

In some embodiments, the neurons show neuron activity on day 25.

As with other neurons, progenitor cells are typically preferred for making assembloids. In some embodiments, SN progenitors are used on day 12 of their differentiation (similar to day 14 symNblasts), where they can be mixed with progenitors of desired organs in the ratios and timepoints specified above.

### 3. Methods of Cell Selection and Characterization

In some embodiments, the method includes steps of selection for and/or isolation of one or more types of neurons. Such steps can include, but are not limited to, FACS or immunopanning.

Immunopanning (Sloan, et al., Neuron 95, 779-790.e6 (2017), specifically incorporated by reference herein in its entirety) is a gentle antibody-based purification technique that can be used to segregate the target neuron(s) from other cells including, but not limited to, non-target neurons and progenitor cells. This method allows the binding of specific cells from a mix to a dish pre-coated with antibodies against cell surface proteins. The cells of interest attach to the antibody and the following wash and dissociation steps are much gentler compared to FACS. Suitable cells markers that can be targeted by antibodies for immunopanning known in the art. Preferably the cell marker is an extracellular marker, when it is the target of immunopanning.

Additionally, or alternatively, the cells characterized, e.g., by analyzing expression (e.g., mRNA or protein) of cell specific markers. Although extracellular markers are preferred to cell selection, extracellular or intracellular markers can be used to characterize cells.

Markers of pluripotent stem cells, partially differentiated and differentiated parasymNs neurons, SCs, SCPs, symNs, SNs and subtypes thereof that can be used to distinguish cells types are discussed herein and are known in the art.

See, e.g., U.S. Published Application Nos. 2022/0195386 and 2019/0093074 each of which is specifically incorporated by reference herein in its entirety.

Non-limiting examples of stem cell markers include OCT4, NANOG, SOX2, LIN28, SSEA4 and SSEA3.

Non-limiting markers of SCP and/or SCs include SOX10, PAX3/GAP43, iSC, CD56, MPZ, and S100B.

Non-limiting markers for symNs include nicotinic receptor CHRNA3/B4, adrenergic receptor ADRA2A/B2, vesicular monoamine transporter VMAT1/2, tyrosine hydroxylase TH, norepinephrine transporter NET, and PHOX2B.

Non-limiting examples of peripheral sensory neuron markers include Brn3A, peripherin, and ISL1.

Non-limiting examples of proprioceptor markers include TrkC, RUNX3, CDHL1, ETV1, and ETV4. Proprioceptors also express SPP1, Parvalbumin (PVALB), calcitonin-gene related protein (CGRP), and SubP.

Non-limiting examples of nociceptor markers include TrkA and RUNX1. Nociceptors can also express SST, PLXNC1, Substance P (SubP), members of the transient receptor (TRP) family TRPV1 and TRPV2 (expressed in medium- to large-diameter nociceptors), the temperature sensitive receptor TRPM8, the cold-activated receptor TRPA1, SCN8A-11A genes (encode Na+ channels Nav1.6-9) and the ATP-activated receptor P2X3 were also investigated.

Non-limiting examples of mechanoreceptor markers include TrkB and RET. Mechanoreceptors also express the mechanically-activated K+ channels TREK-1 (KCNK2) and TRAAK (KCNK4), the acid-sensing ion channels (ASIC1-3) expressed in the Meissner corpuscles and Merkel cells, NF200 (expressed in myelinated A-Bfiber neurons), PIEZO2 (expressed in C-low threshold mechanoreceptors), NECAB2, and FAM19A1 (expressed in low threshold mechanoreceptors).

Non-limiting examples of central nervous system (CNS) markers include PAX6, NESTIN, Vimentin, FOXG1, SOX2, TBR1, TBR2 and SOX1. Non-limiting examples of neuronal cell markers include TUJ1, MAP2, NFH, BRN3A, ISL1, TH, ASCL1, CHAT, PHOX2B, PHOX2A, TRKA, TRKB, TRKC, 5HT, GABA, NOS, SST, TH, CHAT, DBH, Substance P, VIP, NPY, GnRH, and CGRP.

Non-limiting examples of mesenchymal precursor markers are SMA, Vimentin, HLA-ABC, CD105, CD90 and CD73.

Non-limiting examples of Cranial Neural Crest (CNC) markers include PAX6, NESTIN, Vimentin, FOXG1, SOX2, TBR1, TBR2 and SOX1. Non-limiting examples of Melanocyte-competent Neural Crest (MNC) markers include PAX6, NESTIN, Vimentin, FOXG1, SOX2, TBR1, TBR2 and SOX1.

In some embodiments, the method includes steps to drive the mixed SN population to a particular subtype(s), and/or includes a subtype(s) isolation step such as FACS or immunopanning.

For example, a dramatic enrichment (80%) of TRKB+ mechanoreceptors can be induced when the cells are replated at day 6. Thus, in some embodiments, methods of making and enriching for mechanoreceptors include a replating at any one of days 4-12, preferably between days 4-8, most preferably on or about days 5, 6, or 7.

### B. Preparation of Tissue Progenitor Cells

The disclosed assembloids include non-neuron progenitor cells (also referred to herein as tissue progenitor cells). Such tissue progenitor cells include, but are not limited to, progenitors of any tissue that can be innervated by neurons, particularly symN, parasymN, and/or SN. Examples include, but are not limited to, heart, lung, kidney, liver, salivary glands, skin, and gastro-intestinal cells, etc.

Non-limiting examples of tissue progenitor cells and methods of making the same are provided.

### 1. Cardiomyocyte (CM) Progenitor Cells

In some embodiments, the tissue progenitor cells are or include CN. Methods of making CM progenitor cells are known in the art. See, for example, Lin and Zou, “Differentiation of Cardiomyocytes from Human Pluripotent Stem Cells in Fully Chemically Defined Conditions,” STAR Protoc. 1 (1): 100015. (2020), doi: 10.1016/j.xpro.2020.100015; Burridge, et al., “Chemically defined generation of human cardiomyocytes,” Nat. Methods. 2014; 11:855-860; Lian, et al., “Robust cardiomyocyte differentiation from human pluripotent stem cells via temporal modulation of canonical Wnt signaling”, Proc. Natl. Acad. Sci. U S A, 109: E1848-E1857 (2020), and Lin, et al., “Heparin promotes cardiac differentiation of human pluripotent stem cells in chemically defined albumin-free medium, enabling consistent manufacture of cardiomyocytes,” Stem Cells Transl. Med., 6:527-538 (2017), each of which is specifically incorporated by reference herein in its entirety.

A non-limiting method is utilized in the experiments below. On day −1, hPSCs were replated by EDTA at a Matrigel (Corning, 1:20)-coated plates at 250×10³ cells/cm². The next day (day 0), medium was changed to CDBM base medium: DMEM/F12 (Gibco, 11320033), 64 mg/L ascorbic acid (Sigma, A8960), 13.6 μg/L sodium selenium (Sigma, S5261), 10 μg/ml transferrin (Sigma, T3309) and Chemically Defined Lipid Concentrate (Gibco, 11905031). On day 0, 5 μM CHIR99021 was added to CDBM medium. On day 1/5/6, 0.6 U/ml heparin (STEMCELL Technologies, 07980) was added to CDBM medium. On day 2/3/4, 0.6 U/ml heparin and 3 μM XAV were added to CDBM medium. Cardiac progenitors should be ready on day 7. In the original protocol, spontaneous beating should be observed after day 10 without dissociation.

### 2. Lung Progenitors

Lung organoids can be generated in the following steps: endoderm induction in 3D embryonic body (EB) formation, anterior foregut endoderm induction, and formation of lung bud organoids. The assembly with day 14 sympathetic neuroblast can be performed in the last dissociation steps when the progenitor cell fate of target organoids are induced. In the case of lung organoids, endoderm EBs have to be fully dissociated in order to form the anterior foregut endoderm, and sympathetic neuroblasts can be incorporated at this stage. See, e.g., Chen, et al., Nat Cell Biol. 2017 May; 19 (5): 542-549, doi: 10.1038/ncb3510

### 3. Kidney Progenitors

Methods of making kidney progenitor cells are known in the art and can be used in the disclosed assembloids and methods of making the same. See, for example, Zeng, et al., Nat Commun 12, 3641 (2021). doi.org/10.1038/s41467-021-23911-5, which is specifically incorporated by reference in its entirety.

For kidney organoids, symNblast may be incorporated with day 7 dissociated kidney progenitors. Kidney differentiation starts with the mesendoderm induction, followed by the ureteric bud (UB) differentiation, which is the primitive structure of the kidney. The UB differentiation is finished by day 7. Day 7 UBs can be dissociated, and proceed the differentiation of the collecting duct, which is the main functional structure in the kidney.

### 4. Other Progenitors

Methods of making other non-limiting progenitors are also known and can be used in the disclosed assembloids and methods as discussed herein.

For example, methods of making gastric progenitors are also known in the art and can be used in the disclosed assembloids and methods of making the same. See, for example, Broda, Nat Protoc 14, 28-50 (2019). doi.org/10.1038/s41596-018-0080-z, which is specifically incorporated by reference in its entirety.

Methods of making salivary gland progenitors are also known in the art and can be used in the disclosed assembloids and methods of making the same. See, for example, Tanaka, et al., Nat Cell Biol 24, 1595-1605 (2022). doi.org/10.1038/s41556-022-01007-6, which is specifically incorporated by reference in its entirety.

### IV. Assembloid and Compositions Thereof

The technology of human pluripotent stem cell (hPSC)-based 3D organoid/assembloid cultures has become a powerful tool for the study of human embryonic development, disease modeling and drug discovery in recent years. However, the autonomic sympathetic nervous system innervates and regulates almost all organs in the body, including the heart, and yet, most reported organoids to date are not innervated, thus lacking proper neural regulation, and hindering reciprocal tissue maturation. The disclosed methods and compositions solve this problem.

### A. Assembloids

The presently disclosed subject matter provides assembloids and compositions thereof including individual and populations of the same. Typically the assembloids are produced by the in vitro methods described herein. In certain non-limiting embodiments, the neuron progenitor cells and/or tissue progenitor cells of the assembloids are prepared from embryonic pluripotent stem cells, such as human embryonic pluripotent stem cells. In certain non-limiting embodiments, the neuron progenitor cells and/or tissue progenitor cells of the assembloids are prepared from induced pluripotent stem cells, such as induced human pluripotent stem cells. Typically the assembloids include innervation of one or more neuron into a non-neuron tissue (e.g., organoid).

Compositions including one or more assembloids are also provided.

The compositions can be for example the one or more assembloids in culture media or other physiologically acceptable solution. In some embodiments, the compositions are present in a container, such as a dish or flask.

### B. Genetically Modified Cells

In some embodiments, the disclosed assembloids do not contain any gene or genetic modification. Additionally or alternatively, the cells can also be free from transfection with nucleic acid constructs. For example, as exemplified below, the assembloid programs described herein can be carried out without gene modification or transgene expression.

However, in some embodiments, the precursor stem cells and or the progenitor cells have been genetically modified and/or include one or more nucleic acid expression constructs. Gene modifications typically refer to modification of the cell's genome and can be induced by any suitable means, e.g., triplex-forming molecules, pseudocomplementary oligonucleotides, CRISPR/Cas, zinc finger nucleases, TALENs, viral mediated integration, etc. These technologies are known in the art can be used to make modifications to the cells ranging from point mutations to deletions and insertions of e.g., expression constructs. Additional or alternative, the cells may optionally be transfected with transient or permanently nucleic acid expression constructs in the form of e.g., mRNA, viral vectors, plasmids, and other extrachromosomal means of gene expression. Such genetic modifications and expression constructs can be used for a variety of purposes including, but not limited to, facilitating or enhancing preparation of the precursor (e.g., stem) cells, preparation of the differentiated cells (e.g., sensor neurons), and/or for gene therapy.

In particularly preferred embodiments containing a genetic modification or extrachromosomal expression construct, the cells are enhanced for use in gene therapy applications. Gene therapy is a technique that modifies a person's genes to treat or cure disease. Gene therapies can work by several mechanisms, for example, (1) replacing a disease-causing gene with a healthy copy of the gene; (2) inactivating a disease-causing gene that is not functioning properly; or (3) introducing a new or modified gene into the body to help treat a disease. For example, in some embodiments, the cells are used to a treat a disease, such a genetic disorder, that includes reduced expression of a wildtype protein and/or expression of mutant protein. Such cells can be modified to, for example, reverse a detrimental genetic mutation and/or express or overexpress a compensatory protein.

Thus, in some embodiments, cells prepared according to the disclosed methods are infected, transfected or otherwise modified to express an expression construct. For example, constructs can be inserted into vectors for expression in cells. As used herein, a “vector” is a replicon, such as a plasmid, phage, or cosmid, etc., into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence. Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

### C. Conditioned Media

Assembloids may secrete molecules into the culture that may have therapeutic or other effects. Thus, media conditioned by the disclosed assembloids, further compositions formed therefrom, and methods of use thereof are also provided.

Conditioned media can include one or more of the collection of proteins that contain a signal peptide and are processed via the endoplasmic reticulum and Golgi apparatus through the classical secretion pathway, proteins shed from the cell surface, intracellular proteins released through non-classical secretion pathway, extracellular vesicles including but not limited to exosomes. These secreted materials include numerous enzymes, growth factors, cytokines and hormones or other soluble mediators. Conditioned media may influence, e.g., cell growth, differentiation, invasion and angiogenesis by regulating cell-to-cell and cell-to-extracellular matrix interactions of the same or different cell types. In some embodiments, conditioned media from assembloids can be used to rescue a deficiency in a subject in need thereof, e.g., caused by a loss or dysfunction of the corresponding in vivo tissue, particularly where cell therapy is not necessary, or is impractical, or impossible.

The conditioned media can be prepared by culturing a sufficient number of assembloids for a sufficient period of time to secrete the desire factors at the desired amount to achieve the goal, e.g., therapeutic goals. In some embodiments, the culturing is carried out for 1, 2, 3, 4, 5, 6, 7, 8, 9, 10, or more days. The conditioned media can be used without modification (e.g., neat), or the factors therein can be concentrated and/or sub-fractioned.

### D. Compositions

Compositions including assembloids and/or conditioned media made therefrom are also provided. In certain non-limiting embodiments, the composition includes a biocompatible scaffold or matrix, for example, a biocompatible three-dimensional scaffold that facilitates tissue regeneration when the cells are implanted or grafted to a subject. In certain non-limiting embodiments, the biocompatible scaffold includes extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition is a pharmaceutical composition that includes a pharmaceutically acceptable carrier, excipient, diluent or a combination thereof. In certain embodiments, the compositions can be used to treat or prevent a disorder or a disease.

The compositions can be provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the presently disclosed cells.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions should be selected to be chemically inert and will not affect the viability or efficacy of the presently disclosed cells and/or active agents in the conditioned media. This will present no problem to those skilled in chemical and pharmaceutical principles, or problems can be readily avoided by reference to standard texts or by simple experiments, from this disclosure and the documents cited herein.

### E. Exemplary Assembloids

Cardiovascular disease affects millions of people worldwide, and costs billions to the health care systems¹³. The most well-known and life-threatening cardiovascular diseases, such as heart failure and heart attack, may happen when blood supply is not regulated properly¹³. In addition to genetic heart defects, risk factors for heart failure and heart attack include metabolic conditions, such as diabetes and obesity, hypertension, viral infection, drug abuse, smoking, and excessive alcohol intake¹³. Many of these risk factors are mediated by the sympathetic nervous system (SNS). The SNS belongs to the autonomic nervous system and regulates various cardiovascular functions, including heart rate, blood pressure, blood glucose, and gland secretion¹⁴. SymN hyperactivity and neuropathy have been associated with the pathology of diabetes and obesity, hypertension, viral infection, drug abuse, smoking, and alcohol toxicity in adults¹⁵⁻²⁰. Furthermore, symNs innervate the heart as early as the embryonic stage (about E13.5), which may involve in hyperplastic to hypertrophic transition at neonatal stage and cardiac maturation²¹⁻²³. Current studies have shown that developmental disorders that lead to aberrant symN innervation to the heart will cause heart rate variability and arrythmia, which increase the risk of sudden cardiac death²⁴⁻²⁷.

The disclosed compositions and methods provided is an easy to reproduce and cost-effective method without the need for bioengineering or special instrumentation to create sympathetic neuron (symN)-innervated cardiac assembloids. In the experiments below, cardiac tissues were innervated by symNs and showed structural and functional maturation. Thus, sympathetic neuron (symN)-innervated cardiac assembloids are provided.

SymNs in the assembloids were functional and were able to regulate cardiac contraction and maturation. Hypoxic stress induced endogenous NE over secretion, and synergistically led to cardiac infarction features in the assembloids.

Thus, in some embodiments, the human sympathetic cardiac assembloids (hSCAs) show mature muscle structures, atrial to ventricular patterning, and/or spontaneous beating. In some forms hSCA-innervating symNs display neurotransmitter synthesis and/or functional regulation of the cardiac beating rate, which can be manipulated pharmacologically or optogenetically. In some forms, symN-mediated cardiac development and myocardial infarction can be modeled. These hSCAs provide a tool for neurocardiotoxicity screening approaches and is highly versatile and modular, where the type of neuron (symN or parasympathetic or sensory neuron) as well as the type of organoid (heart, lung, kidney, etc.) to be innervated may be interchanged. Thus, this symN-heart axis assembloid paradigm therefore provides not just an ideal platform for assessing the mechanisms that environmental factors exert on cardiac function through sympathetic regulation, but also a playbook for generating innervated organoids from other organ systems.

### V. Methods of Use

Methods of using the disclosed assembloids and compositions thereof are also provided. The methods include investigation of disease etiology, treatment of diseased subjects, and testing of compounds for e.g., therapeutic or toxic effect.

### A. Methods of Treatment

In certain embodiments the assembloids can be used for preventing and/or treating a disorder. In some embodiments, the disease or disorder one of the tissue or organ corresponding to the assembloid. Such methods can include administering to a subject in need thereof a therapeutically effective amount of the presently disclosed assembloids, conditioned media, or a composition form therefrom.

The assembloids or compositions thereof can be administered or provided systemically or locally to a subject for preventing and/or treating the disease or disorder. In certain embodiments, cells, conditioned media, or a composition formed therefrom are directly injected into or grafted to or adjacent to an organ of interest.

The cells and compositions thereof can be administered in any physiologically acceptable vehicle. Pharmaceutical compositions cell or a composition thereof and a pharmaceutically acceptable carrier are also provided. In some embodiments, the cells or compositions are administered via localized orthotropic (OT) injection, local application, systemic injection, intravenous injection, or parenteral administration.

The cells, media, or a composition thereof can be administered to a subject in a therapeutically effective amount. A “therapeutically effective amount” is an amount sufficient to affect a beneficial or desired clinical result upon treatment. A therapeutically effective amount can be administered to a subject in one or more doses. In terms of treatment, a therapeutically effective amount is an amount that is sufficient to palliate, ameliorate, stabilize, reverse or slow the progression of the disorder of neurons and/or neurodegenerative disorder, or otherwise reduce the pathological consequences of the disease or disorder. The therapeutically effective amount is generally determined by the physician on a case-by-case basis and is within the skill of one in the art. Several factors are typically taken into account when determining an appropriate dosage to achieve a therapeutically effective amount. These factors include age, sex and weight of the subject, the condition being treated, the severity of the condition and the form and effective concentration of the cells administered.

The quantity of cells or amount of conditioned media to be administered will vary for the subject being treated and the condition being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the presently disclosed cells are administered to a subject. The precise determination of what would be considered a therapeutically effective dose may be based on factors individual to each subject, including their size, age, sex, weight, and condition of the particular subject. Dosages can be readily ascertained by those skilled in the art from this disclosure and the knowledge in the art.

In some embodiments, the cells are transiently or genetically modified as discussed above. Such cells can serve as a combination of both cell and gene therapy. The appropriate gene modification and/or recombinant expression construct can be selected by the practitioner based on the disease or disorder to be treated. For example, in some embodiments the cells are modified to enhance treatment of a monogenic disorder. Monogenic disorders (monogenic traits) are caused by variation in a single gene and are typically recognized by their striking familial inheritance patterns. In such embodiments, the genetic modification may reverse or correct or otherwise compensate for the mutated gene by, for example, expressing a wildtype copy of the mutant gene.

A non-limiting example is Familial Dysautonomia (FD). FD is a monogentic disorder that is caused by a homozygous point mutation in the ELP1 gene. This leads to missplicing of the elongator complex protein 1 (ELP1) protein. The mutant protein is degraded, and patients thus have dramatically reduced levels of ELP1 protein. This is particularly prevalent in sensory and sympathetic tissues. Thus, in some embodiments, the cells are genetical modified to express one or more wildtype copies of ELP1 protein, e.g., by having one or more endogenous gene and/or heterologous expression constructs that encode and express wildtype ELP1 protein (e.g., an active copy of the ELP1 gene). Accordingly, for example, ELP1-gene delivery into hPSC-derived parasymN and transplanted into the DRG of patients may be used as a therapy for FD.

In other embodiments, the compositions are used to treat an autoimmune disorder, particularly where parasympathetic neurons are attached, e.g., Sjörgen's syndrome.

In other embodiments, the compositions are used to treat an infectious disease, e.g., that cause neural damage or dysfunction, e.g., to parasympathetic neurons, including, but not limited to SARS-COV-2.

In other embodiments, the compositions are used in therapeutic or non-therapeutic methods of enhancing development and/or maturation of tissue that is innervated by neurons, e.g., cardiovascular tissue, adipose tissue, etc. In some embodiments, the compositions are used to regulate white adipose tissue (WAT) maturation and functionality, for example, by negatively regulating WAT lipolysis and/or decreasing glucose uptake in adipocytes.

### B. Drug Screening

The disclosed cells and compositions thereof are useful to investigate the activity or applicability of one or more test compounds to treat or alleviate one or more symptoms of a disease or disorder.

In a typical embodiment, assembloids are formed as disclosed herein. In some embodiments, the starting cells (e.g., stem cells) are isolated from a diseased subject, or healthy cells are treated with a disease-inducing compound, to form a diseased, dysfunctional, or a defective cell model. One or more test compounds can be applied to cultured assembloids and evaluated for the ability to treat one or more symptoms of the diseased, dysfunctional, or defective cells. The symptom or symptoms can be specific to the disease state being studied, or can be of a generally nature.

In some embodiments, healthy cells are utilized, and compounds are tested for toxicity and/or the ability to further improve one or more wildtype functions.

Physiological, phenotypic, morphological, or molecular symptoms and other markers of the cells can be monitored over time.

### C. Diseases and Disorders

Diseases and disorders that can be the subject of the disclosed methods include those that effect neurons and may be neurodevelopmental and/or neurodegenerative. In some embodiments the disease or disorder is a condition of parasympathetic nervous system (PSNS), an autonomic neuropathy, neurodegenerative disease, autoimmune disease, infectious disease, developmental disease or disorder, or injury.

Exemplary PSNS and autonomic diseases and disorders include, but are not limited to, type 2 diabetes, congenital and genetic conditions including but not limited to amyloidosis and Familial Dysautonomia, loss of control of bladder and/or bowels, multiple system atrophy, sexual dysfunction (e.g., erectile dysfunction), orthostatic hypotension, postprandial hypotension, pure autonomic failure, afferent baroreflex failure, and trauma.

Exemplary neurodegenerative diseases include, but are not limited to, amyotrophic lateral sclerosis (ALS), Huntington's disease (HD), and muscular dystrophies, problems with the way the nervous system develops, such as Spina bifida, degenerative diseases, where nerve cells are damaged or die, such as Parkinson's disease (PD) and PD-related disorders, meningitis, prion diseases such as Creutzfeldt-Jakob Disease, corticobasal degeneration, frontotemporal dementia, cognitive impairment including mild cognitive impairment and HIV-related cognitive impairment, motor neuron diseases (MND), spinocerebellar ataxia (SCA), spinal muscular atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, monomelic amyotrophy, multiple system atrophy, multiple system atrophy with orthostatic hypotension (Shy-Drager Syndrome), Multiple Sclerosis (MS), neurodegeneration (e.g., with brain iron accumulation), opsoclonus myoclonus, posterior cortical atrophy, primary progressive aphasia, progressive supranuclear palsy, vascular dementia, progressive multifocal leukoencephalopathy, dementia with Lewy Bodies, lacunar syndromes, hydrocephalus, Wernicke-Korsakoff's syndrome, post-encephalitic dementia, cancer and chemotherapy-associated cognitive impairment and dementia, and depression-induced dementia, Guillain-Barré syndrome, and pseudodementia.

In some embodiments, the disease or disorder is not primarily or exclusively a central nervous system (CNS) disease or disorder.

The results below show that parasympathetic neurons may be a direct target of autoimmune diseases, particularly where autoantibodies are directed against parasymN marker such as a MusR. The results also show that parasymN have an anti-inflammatory activity, particularly against TNF-α and IL-6 released by immune cells. Thus, in some embodiments, an autoimmune disease is treated, particularly one in which parasympathetic neurons are directed targeted by the disease such as Sjogren's syndrome. In other embodiments, an inflammatory disorder or condition is treated, particularly one characterized by increased TNF-α and IL-6, such following infection, e.g., with SARS-COV-2.

Representative inflammatory or autoimmune diseases and disorders that may be treated include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopeniarpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis-juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, Wegener's granulomatosis, and infectious diseases, including, but not limited to bacterial and viral infections such as SAR-CoV-2, and the diseases associated therewith (e.g., COVID).

In some embodiments, the compositions and methods are used to improve heath and developed, e.g., of the nervous system, particularly the parasympathetic nervous system. Such methods may be therapeutic or none therapeutic.

The invention can be further understood by the following numbered paragraphs:
1. A method of making assembloids including combining dissociated neuron progenitor cells with dissociated tissue progenitor cells and culturing them under free floating 3D culture conditions suitable for the neuron progenitor cells and tissue progenitor cells to form one or more assembloids.
2. The method of paragraph 1, wherein the assembloids are cultured under suitable conditions and duration for the neuron progenitor cells and tissue progenitor cells to mature.
3. The method of paragraph 2, wherein maturation includes expression of one or more markers; presence of one or more structures and/or ultrastructures; and/or one or more functionalities consistent with the corresponding mature tissue or system in vivo.
4. The method of any one of paragraphs 1-3, wherein the method is carried out free from special instruments and/or scaffolds, optionally wherein the special instrument and/or scaffold is or includes a bioprinter, hydrogel, and/or organ-mimicking scaffold.
5. The method of any one of paragraphs 1-4, wherein the neuron progenitor cells and tissue progenitor cells are mixed under movement or agitation, optionally using a shaker optionally wherein the shaker is an orbital shaker.
6. The method of any one of paragraphs 1-5, wherein the neuron progenitor cells and tissue progenitor cells, and/or assembloids are cultured under movement or agitation, optionally using a shaker optionally wherein the shaker is an orbital shaker.
7. The method of any one of paragraphs 1-6, wherein the neuron progenitor cells are sympathetic neuron progenitor cells, parasympathetic neuron progenitor cells, and/or sensory neuron progenitor cells, optionally wherein the sensory neuron progenitor cells are nociceptors, mechanoreceptors, and/or proprioceptors.
8. The method of any one of paragraphs 1-7, wherein the tissue progenitor cells are heart, lung, kidney, liver, salivary gland, skin, and/or gastro-intestinal progenitor cells.
9. The method of any one of paragraphs 1-8, wherein the neuron progenitor cells are symNblast sympathetic neuron progenitor cells prepared from human pluripotent stem cells.
10. The method of any one of paragraphs 1-9, wherein the tissue progenitor cells are day 7 cardiomyocytes prepared from human pluripotent stem cells.
11. An assembloid formed according to the method of any one of paragraphs 1-10.
12. A 3D assembloid including neurons innervating a second or more tissues.
13. The assembloid of paragraphs 11 or 12 wherein the neurons include sympathetic neurons, parasympathetic neurons, and/or sensory neurons, optionally wherein the sensory neurons are nociceptors, mechanoreceptors, and/or proprioceptors.
14. The assembloid of any one of paragraphs 11-13, wherein the second or more tissues include heart, lung, kidney, liver, salivary gland, skin, and/or gastro-intestinal cells.
15. An assembloid including sympathetic neurons and cardiac cells.
16. A composition including the assembloid of any one of paragraphs 11-15.
17. The composition of paragraph 16 including culture media.
18. Conditioned media formed by culturing assembloid(s) of any one of paragraphs 11-15.
19. A method of determining the effect of a compound including measuring one or more characteristic(s) of cultured assembloid(s) of any one of paragraphs 11-15 a first time, contacting the assembloid(s) with the compound, and measuring the characteristic(s) of the cultured assembloid(s) a second time.
20. The method of paragraph 19, wherein a plurality of compounds are separately contacted with a plurality of separately cultured assembloids, optionally wherein the assembloids are separately cultured in a multiwell plate or dish.

### EXAMPLES

### Methods

hPSC Maintenance

Human embryonic stem cell (hESC) line WA09 (H9) and optogenetic iPSC line hiPSC (ChR2/NpHR)⁵² were mainly used in this study. hPSCs were maintained in Essential 8 medium (Gibco, A15170-01) on vitronectin coated (Thermo Fisher/Life Technologies, A14700, 5 μg/ml) cell culture plates, and passaged using EDTA (Sigma, ED2SS) as previous descrobed³⁰.

hSCA Differentiation

SymNblast. A detailed differentiation protocol can be found in a previous publication³⁰. Day 0: hPSCs were dissociated by EDTA and replated on Geltrex (Invitrogen, A1413202)-coated plates at 125×10³ cells/cm². Cells were fed with day 0-1 medium: Essential 6 medium (Gibco, A15165-01), 0.4 ng/ml BMP4 (PeproTech, 314-BP), 10 μM SB431542 (R&D Systems, 1614) and 300 nM CHIR99021 (R&D Systems, 4423). From day 2 on, cells were fed with day 2-10 medium: Essential 6 medium, 10 μM SB431542 and 0.75 μM CHIR99021. For the best result, BMP4 titrations can be performed for each batch/lot due to the batch-to-batch variability of BMP4. NC cell fate should be induced by day 10. Day 10 NCCs were dissociated by accutase (Coring, AT104500) and replated to ultra-low attachment plates to form symNblast spheroids. The symNblast medium for day 10-14 contains Neurobasal medium (Gibco, 21103-049), B27 (Gibco, 17502-048), N2 supplement (Thermo Fisher/Gibco, 17502048). L-Glutamine (Thermo Fisher/Gibco, 25030-081), 3 μM CHIR99021 and 10 ng/ml FGF2 (R&D Systems, 233-FB/CF).

CM progenitor. A detailed differentiation protocol is described by Lin et al.³⁶. On day −1, hPSCs were replated by EDTA at a Matrigel (Corning, 1:20)-coated plates at 250×10³ cells/cm². Next day on (day 0), medium was changed to CDBM base medium: DMEM/F12 (Gibco, 11320033), 64 mg/L ascorbic acid (Sigma, A8960), 13.6 μg/L sodium selenium (Sigma, S5261), 10 μg/ml transferrin (Sigma, T3309) and Chemically Defined Lipid Concentrate (Gibco, 11905031). On day 0, 5 μM CHIR99021 was added to CDBM medium. On day 1/5/6, 0.6 U/ml heparin (STEMCELL Technologies, 07980) was added to CDBM medium. On day 2/3/4, 0.6 U/ml heparin and 3 μM XAV were added to CDBM medium.

hSCA assembly. Day 14 symNbalsts and day 7 cardiac progenitors were dissociated by accutase. Cells were fully mix at 1:1 ratio as 100×10³ symNblasts +100×10³ cardiac progenitors on 96-well ultra-low attachment plates to form one organoid per well, or as 500×10³ symNblasts+500×10³ cardiac progenitors on 24-well ultra-low attachment plates for bulk organoid generation (about 5 organoids will form per well). From now on, assembloids were fed with hSCA medium: Neurobasal medium+CDBM base at 1:1 ratio, 100× B27, 200× L-Glutamine, 200× N2, 12.5 ng/ml GDNF, 12.5 ng/ml BDNF, 12.5 ng/ml NGF, 100 μM ascorbic acid, 100 μM dbcAMP, 0.0625 μM retinoic acid (add RA freshly every feeding), and 10 μg/ml insulin (Sigma, I-034). hSCAs were fed every 3 days.

### RT-qPCR

0.5×10⁶ cells were collected using Trizol (Invitrogen, 15596026) for each sample. Reverse transcription with 1 μg total RNA was performed using iScript™ Reverse Transcription Supermix (Bio-Rad, 170884). SYBR green (Bio-Rad) RT-qPCR was ran using CFX96 Touch Deep Well Real-Time PCR Detection System (Bio-Rad), and analyzed by CFX Maestro.

### Image Based Beat Quantification

hSCA beating was analyzed using the ImageJ Time Series Analyzer plugin (Balaji J. UCLA). hSCA beating video was saced as AVI file for the analysis.

### Multielectrode Array (MEA)

To measure cardiac signaling, hSCAs were placed on dry MEA plates (Axion BioSystems, BioCircuit or CytoView), one organoid per 96 well. After sitting for a few minutes, hSCA medium was added on top of the slightly attached organoids in 20-50 μl droplets. Do not fully cover the entire bottom area of the well to prevent the organoids from floating. hSCA field potential was measured using a MEA plate reader (Axion BioSystems, Maestro Pro) under the cardiac detection mode according to manufacturer's instruction.

### Cryo-Sectioning

hSCAs were fixed by 4% paraformaldehyde overnight and washed twice by PBS. Fixed hSCAs were dehydrated by 30% sucrose in PBS overnight until the organoids sink to the bottom. hSCAs were then embedded in OCT compound (Sakura Finetek) and cryo-sectioned at 7 μm thickness.

### Immunohistochemistry

Cryo-section staining. Sections were permeabilized and blocked by 0.2% Triton X-100 and 3% goat or donkey serum in PBS for 60 minutes, and incubated with primary antibodies in the blocking buffer overnight at 4° C. Sections were rinsed twice by PBS incubated with secondary antibodies in the blocking buffer overnight at room temperature (RT). After PBS wash, sections were mounted using mounting medium with DAPI (Abcam, ab104139).

Whole mount staining. Fixed hSCAs were permeabilized by 0.3% Triton, 1% BSA and 3% goat or donkey serum in PBS for 2-4 hours at RT. Primary antibodies were added and incubated for 48 hours at 4° C. After PBS wash for one hour at RT, secondary antibodies were added and incubated for 24 hours at 4° C. After PBS wash for one hour at RT, DAPI was added and incubated for one hour at RT.

Fluorescent images were taken using the Lionheart FX Automated Microscope. Primary and secondary antibodies used in this study were listed in Table 1.

### Wheat Germ Agglutinin (WGA) Staining

hSCA cryo-sections were incubated with 5 μg/ml 488-conjugated WGA (biotium, 29022) for 30 minutes at RT. Permeabilization was performed afterward and regular immunostaining was performed after WGA staining.

Phalloidin staining (for F-actin)

Phalloidin-iFluor 488 Reagent (Abcam, ab176753, 1:1000) was used according to manufacturer's instructions. Cryo-sectioned hSCAs were permeabilized and blocked by 0.2% Triton X-100 and 3% goat or donkey serum in PBS for 60 minutes, then incubated with 1× Phalloidin solution in PBS with 1% BSA for 30 minutes. Sections were washed by PBS for at least 3 times. General immunohistochemistry staining can be performed after Phalloidin staining.

### Norepinephrine Live Labeling

hSCAs were incubated with 1 μM NE tracer NS510 in hSCA culture medium and at 37° C. for 60 min. After PBS wash for at least twice, hSCAs were imaged using Lionheart FX Automated Microscope at 440 nm excitation and 520 nm emission.

### NE ELISA

NE assay was performed according to manufacturer's instructions (EagleBio, NOU39-K01). hSCA lysates from 1 well of 24-well plate were collected in 200 μl PBS. To preserve NE, sample stabilizer included in the kit was added to each sample. Lysate solutions were spun at 300×g for 5 min to remove debris. The samples were ready for NE detection or were stored at −80° C. for long-term storage (although not recommended).

### Light Sheet Microscopy

The imaging of the hSCAs was conducted using a custom-built light-sheet microscope⁷⁵. The microscope was outfitted with a 16×/0.8 NA water-immersion detection objective (Nikon N16XLWD-PF). For the excitation of GFP fluorescence, a 488 nm laser was employed, operating at a power density of 1.86 W/cm². Similarly, a 561 nm laser was used to stimulate RFP fluorescence, also at a power density of 1.86 W/cm². The light sheet's thickness at the beam waist was 6.7 μm. An exposure time of 50 ms was maintained during imaging. The volumetric images captured were 100×276.48×276.48 μm³ in size (200×2048×2048 pixels), offering a resolution of 1.64×0.313×0.313 μm. For sample preparation, hSCAs were immobilized in a solution of 1% low melting point agarose (Sigma) with 1% PBS (Gibco), subsequently placed within a petri dish for stable imaging.

### Transmission Electron Microscopes (TEM)

hSCAs were fixed in Trump's EM fixative: [4% paraformaldehyde, 1% glutaraldehyde in 0.1M Phosphate buffer, pH 7.25] and washed several times in 0.1M Phosphate buffer before post-fixation in 1% osmium tetroxide in buffer for 1 hour. The organoid samples were washed several times in deionized water and then placed in a 0.5% aqueous uranyl acetate enbloc for 1 hour in the dark. After several more washes in deionized water, the organoid samples were dehydrated in an ethanol series [30%, 50%, 75%, 95%, 100%], cleared in two changes of acetone, and two changes of propylene oxide. The organoid samples were infiltrated with 2:1, 1:1 and 1:2 mixtures of propylene oxide and Mollenhauer's Epon-Araldite plastic mixture⁷⁶ two hours respectively; then two changes of 100% Epon-Araldite plastic for at two hours each before embedding the tissues in flat embedding molds. The embedded samples were polymerized in a 70-80° C. oven overnight⁷⁷. 1 μm sections from the polymerized blocks were obtained using a Reichert Ultracut S ultramicrotome. Sections were placed on glass slides and stained with 1% Toluidine Blue O in 1% sodium borate. The stained sections were evaluated, and areas of interest were chosen before trimming the corresponding block face for thin sectioning. 60-70 nm sections were obtained and placed on 200-mesh copper Locator grids. One of the grids was post stained with 2% aqueous uranyl acetate and Reynolds lead citrate (Reynolds, 1963), while the remaining grids were left unstained. Grids were viewed with a JEOL JEM-1011 transmission electron microscope at varying magnifications using an accelerating voltage of 100 KeV. Images were acquired using an AMT XR80M Wide-Angle Multi-Discipline Mid-Mount CCD Digital Camera with a resolution of 3296×2460 pixels.

### Calcium Imaging

hSCAs were incubated with 4 μM of Fluo-4 AM (TOCRIS, 6255) in hSCA culture medium and incubated at 37° C. for 30 minutes. After PBS wash for three times, hSCAs were incubated for 30 min with fresh medium at 37° C. After the incubation, hSCAs were read and video was made using Lionheart FX Automated Microscope.

### Optogenetic Stimulation

hSCAs that are consist of hiPSC (ChR2/NpHR)-derived symNs were stimulate by blue light (HQRP) using the High-Accuracy Digital Electronic Timer (GraLab, model 451) in 1 second on, 4 second off frequency for 5 minutes.

### Low Oxygen Model for Cardiac Infarction

Wk5 hSCAs were fed with hSCA medium and placed into the hypoxia chamber (incubator subchamber system with O₂/CO₂ setpoint control, BioSpherix) with 10% O₂, control group was maintained in regular culture condition (21% O₂). hSCAs in both conditions were fed with fresh hSCA medium every 3 days for 10 days.

### Atomic Force Microscopy (AFM)

hSCAs were fixed by 4% PFA for 24 hours before AFM measurement. Stiffness was measured by AFM (Agilent Technologies 5500 Scanning Probe Microscope) using Aluminum coated cantilever (ASPIRE CCSR-10, spring constant=0.1 N/m). Fixed hSCAs were placed on the surface with minimal liquid remaining, and measured immediately. The indentation measurement up to the depth about 200 μm. The force-distance curves collected were fitted with the modified Hertz model to calculate the Young's Modulus.

### Image-iT™ Hypoxia Staining

4 μm Image-iT™ Green Hypoxia Reagent (ThermoFisher, I14833) was given to hSCAs and incubated at 37° C. for 60 minutes. Stained hSCAs were washed by PBS for 3 time and imaged by Lionheart FX Automated Microscope.

### Results

Generation of hSCAs

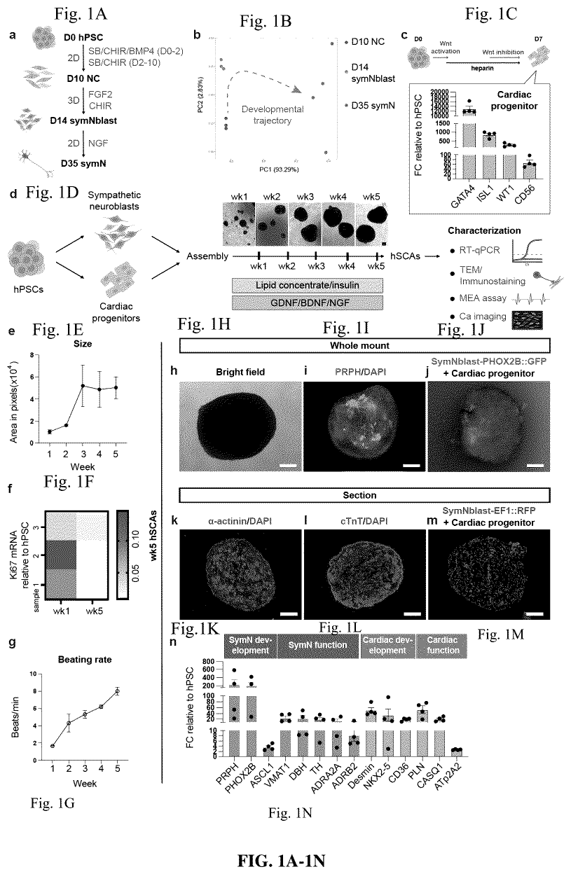

The sympathetic nervous system consists of two neural parts, the preganglionic and postganglionic symNs. While the preganglionic symNs are spinal motor neurons derived from the neuroectoderm, and therefore belong to the central nervous system, postganglionic symNs are the ones that innervate target tissues and communicate via the norepinephrine (NE) neurotransmitter²⁰. Directed differentiation protocol for postganglionic symN have been established (FIG. 1A)^{29,30}. In this protocol, hPSCs are first differentiated into SOX10⁺ neural crest cells (NCCs), the progenitors of peripheral neurons³⁰, in 2D, adherent cultures, with high efficiency (about 90%³⁰). By replating the differentiated NCCs into 3D spheroids, the early sympathetic progenitor markers, PHOX2B, ASCL1, HAND2, GATA2, GATA3, are induced, representing sympathetic neuroblasts (symNblast)^{29,30}. Neurons derived from symNblasts yield high purity (about 80%²⁹) express typical symN markers, including nicotinic receptor CHRNA3/B4, adrenergic receptor ADRA2A/B2, vesicular monoamine transporter VMAT1/2, tyrosine hydroxylase TH, and norepinephrine transporter NET^{29,30}. hPSC-derived symNs are spontaneously firing action potentials, which can be upregulated by nicotine treatment, and they can form connections to CMs in 2D co-cultures^{29,30}. Bulk RNA seq. analysis (Wu et al.,)²⁹ showed that this protocol recapitulates the proper developmental stages, from neural crest to symN progenitors to symNs (FIG. 1B).

The heart consists of multiple cardiac lineages, including smooth muscle cells (of the vasculatures), cardiac epithelial and endothelial cells, and cardiac fibroblasts that produce the extracellular matrix^{33,34}. For hSCAs, a CM differentiation protocol modified from Lin et al.,^{35,36} was used to generate early cardiac progenitors, which may still have the multipotency to be differentiated into several of the cardiac lineages^{37,38}. hPSCs are cultured in 2D, first induced by the WNT activator CHIR99021, followed by WNT inhibition using XAV939. Cardiac progenitors expressed typical markers, including GATA4, WT1, ISLET-1, and CD56 shown by RTqPCR on day 7 (FIG. 1C). To form hSCAs, dissociated symNblasts (day 14) and cardiac progenitors (day 7) were mixed and cultured in low attachment plates, in floating cultures on a shaker for up to 5 weeks (FIG. 1D). The size of the hSCAs increased until week 3 and stabilized after (FIG. 1D-1E), which was in line with decreased Ki67 expression from wk1 to wk5 (FIG. 1f). The hSCAs were beating spontaneously with a beating rate that increased over time (FIG. 1G), and which was quantified via video-based image quantification (FIG. 6). These results indicate that on wk5, hSCAs have stopped proliferating and may have entered their maturation stage.

On wk5, the cellular composition in hSCAs was assessed by whole mount staining. Peripheral neural marker PRPH labeled neurons growing inside the organoids (FIG. 1I). By differentiating symNblast from a hPSC line that carries a PHOX2B-driven GFP reporter, it was confirmed that the neurons in hSCAs are PHOX2B⁺ symNs (FIG. 1J). Upon cryosectioning of the hSCAs, the presence of α-actinin⁺ and cTnT⁺ cardiac cells was confirmed (FIG. 1K, 1L). Using an EF1-driven RFP reporter hPSC line, symNblasts were differentiated, assembled into hSCAs. symNs grew inside the cardiac mass, and were more abundant on the outskirts (FIG. 1M). Such a pattern of neural distribution is similar to the one seen in the living heart21,39. RT-qPCR analysis on wk5 hSCAs detected markers indicating both symN (PRPH, PHOX2B, ASCLI) and cardiac development (PLN, Desmin, NKX2.5), as well as maturation (DBH, ADRA2A/B2, VMAT1, ATpA2, CD36, CASQ1, FIG. 1N).

hSCAs Self-Organization

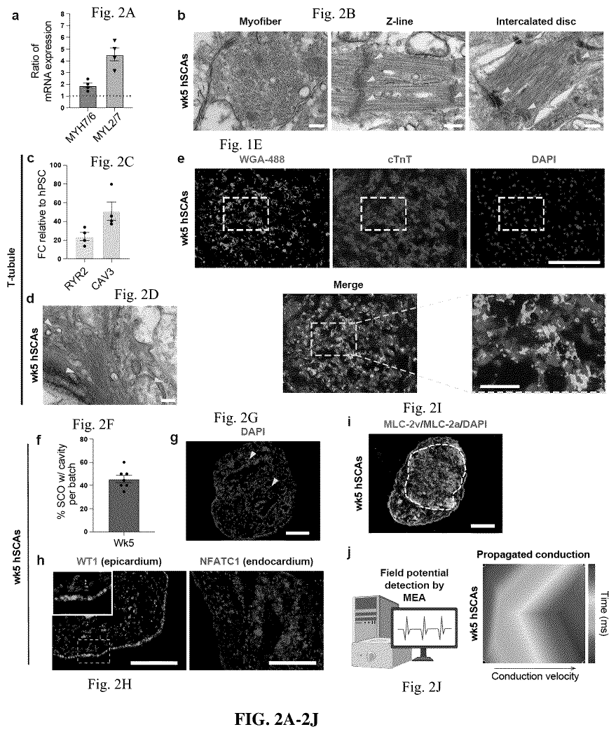

One of the gold standards for a qualified organoid/assembloid is its ability of cellular and structural self-organization, often indicating maturation and functionality⁴⁰. Therefore, the maturity of the cardiac tissue was evaluated within the hSCAs on wk5 via the ratio of MYH7/6 and MYL2/7 expression, which indicate human cardiac tissue maturity if they are above 1⁴¹. In wk5 hSCAs, both MYH7 and MYL2 levels were higher than MYH6 and MYL7, respectively (FIG. 2A). To further examine the hSCAs maturity and organization, wk5 hSCAs were assessed by transmission electron microscopy (TEM). The ultra-structures of well-aligned myofiber bundles, Z-lines, and intercalated discs were observed (FIG. 2B). In addition, transverse tubules (t-tubules) are internalized CM membranes that surround the muscle fibers and are enriched in ion channels, which are important for mature excitation-contraction coupling and heart function^{42,43} RT-qPCR analysis of wk5 hSCAs identified the expressions of T-tubule markers RYR2 and CAVEOLIN3 (FIG. 2C). T-tubule protein structures were detected within the cardiac tissues using TEM (FIG. 2D), and via wheat germ agglutinin (WGA) staining (FIG. 2E).

Interestingly, in about 50% of hSCAs of each differentiation (FIG. 2F), cavity structures were observed (FIG. 2G). Cavities were compartmentalized by a single layer of cells, excluding the possibility that the cavities are caused by necrosis that are often seen in long-term cultured organoids⁴⁴. Similarly, a single cell layer was also formed in the exterior of wk5 hSCAs (FIG. 2G). To assess whether the cavity structures are mimicking heart chambers, hSCAs were stained for epicardium marker WT1 and endocardium marker NFATC1. The results showed that the exterior cell layer was WT1⁺, while the interior layer was TFATC1⁺ (FIG. 2H), indicating both epicardium and endocardium patterning within the hSCAs. Furthermore, hSCAs showed polarized patterning for the atrial marker MLC-2a and ventricular marker MLC-2v (FIG. 2I). Using microelectrode array (MEA), a propagating beating pattern was detected in hSCAs (FIG. 2J). Together, these data indicate that wk5 hSCAs are self-organized in their structure and cell type variety, are relatively mature, and the tissues are functional.

Functional Coupling and Sympathetic Regulation in hSCAs

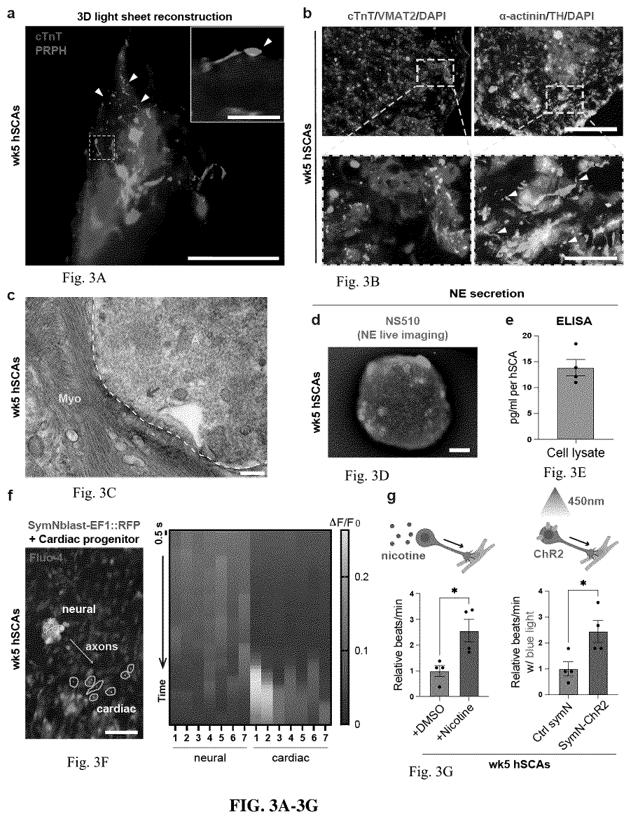

Next, whether the symNs in hSCAs are physically innervating the heart muscles was investigated. Using light sheet microscopy, the 3D muscle mass in hSCAs in high resolution was reconstructed. Using whole mount staining, it was observed that symNs were deeply associated with and growing throughout the cardiac tissues (FIG. 3A). The points of the physical contact between symN axons and cardiac tissues, which form classic swelling and nodal structures^{45,46}, were also identified (FIG. 3A, arrows). The symN axon terminals were then stained with VMAT2 and TH and the co-localized signals of both within the cardiac tissue, as well as the nodal bouton en passant-like structure along the axon terminals was confirmed (FIG. 3B, white arrows). Accordingly, TEM imaging also confirmed the physical innervation of symN axons to CMs in hSCAs (FIG. 3C).

Aside from the physical connection, whether symNs in the assembloids are able to regulate cardiac function was examined. NE is the main neurotransmitter used by symNs and is important for functional regulation of the heart^{20,47}. Thus, if symNs in hSCAs synthesize and release NE was examined. NS510 is a highly sensitive NE chemical probe that has been used to study real-time NE synthesis and dynamics in chromaffin cells and symNs^{29,48}. Using NS510, NE was detected throughout the assembloids (FIG. 3D). The NE level was also detectable in cell lysates by ELISA (FIG. 3E). To assess symN activity, calcium (Ca²⁺) imaging was performed using Fluo-4 Ca²⁺ labeling. To be able to distinguish Ca²⁺ fluxes in symNs and not in cardiac tissues, symNblast were differentiated using the EF1-RFP reporter hPSC line and mixed with unlabeled cardiac progenitors (FIG. 3F). Over time, Ca²⁺ sparks were observed first in symNs (FIG. 3F), followed by sparks in cardiac cells, which are downstream of the axons of the symNs (FIG. 3F). This result demonstrates that both symNs and cardiac tissues in hSCAs are functional, and cardiac activity can be regulated by symN activation. To test if hSCA beating can be manipulated by activating symNs, two methods were used to stimulate symNs within the hSCAs: (1) pharmacological activation using nicotine (1 μM), a widely used method in in vitro symN and CM cocultures that has been shown to exclusively activate symNs in short-term treatment^{29,49-51}. (2) symNblasts were differentiated from an optogenetic iPSC line that expresses ChR2, and can be activated by blue light exposure⁵². Both nicotine treatment and blue light exposure resulted in increased hSCA beating efficiency (FIG. 3G). Together, these data indicate that symNs and cardiac tissues in hSCAs are functionally connected, and that the cardiac tissue can be manipulated by symN activation.

### SymNs Regulate Cardiac Development Through NE Signaling

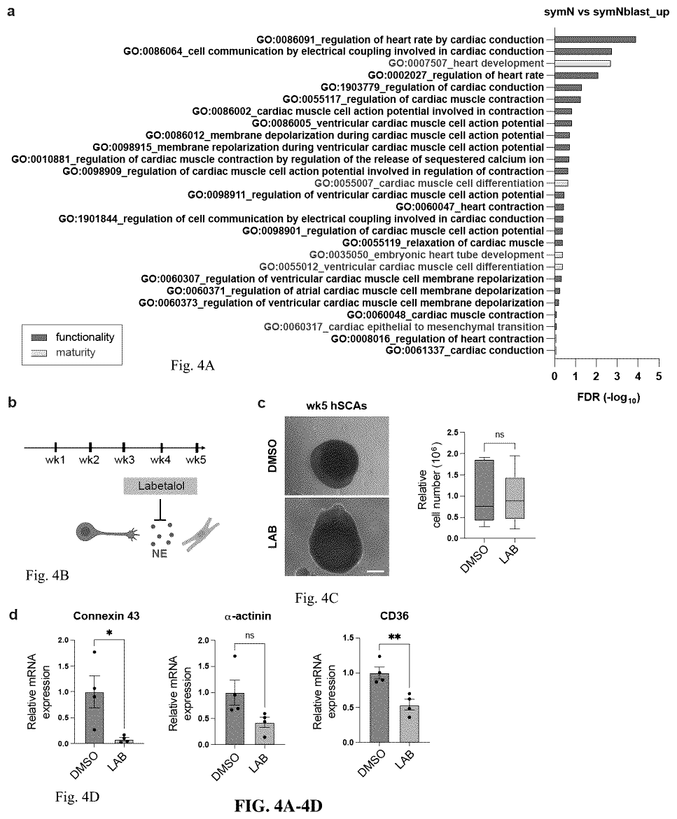

The effect of SymN activation on heart development has been reported, potentially through NE and adrenergic signaling^{22,23}. In vitro 2D co-cultures using hPSC-derived or primary cultured symNs and CMs also demonstrated that symN connectivity facilitates CM maturation⁵³. Bulk RNA seq. data was re-analyzed comparing hPSC-derived symNBlasts (SSRN: dx.doi.org/10.2139/ssrn.4318816) and symNs²⁹, and it was found that during maturation of the neurons alone (FIG. 1A) GO terms of genes that are involved in cardiac development and regulation pathways were significantly upregulated (FIG. 4A), indicating that the neurons acquire the capability to regulate and mature cardiac tissue. To test if the symNs in hSCAs play a regulatory role on cardiac development and maturation, the assembloids were treated with α- and β-adrenergic receptor antagonist labetalol (LAB, 1 μM) to fully block the NE signaling during the growth of the hSCAs (FIG. 4B). The drug was given from wk3, the stage when the assembloid growth almost reaches its plateau (FIG. 1E). Given that symN activity may promote cellular hyperplastic to hypertrophic transition of CM^{22,23}, the overall size of hSCAs was compared on wk5 after LAB treatment. Differences in assembloid size were not observed between DMSO or LAB treated hSCAs (FIG. 4C). However, RT-qPCR analysis for markers of CM maturity^{53,54} (CX43, α-ACTININ, and CD36) revealed decreased expressions upon LAB treatment (FIG. 4D). This result indicates that symNs promote cardiac development through NE signaling in hSCAs; however, NE signaling alone may not be responsible for cardiac hypertrophic transition.

hSCAs Model Hypoxia-Induced Cardiac Infarction Through Endogenous NE

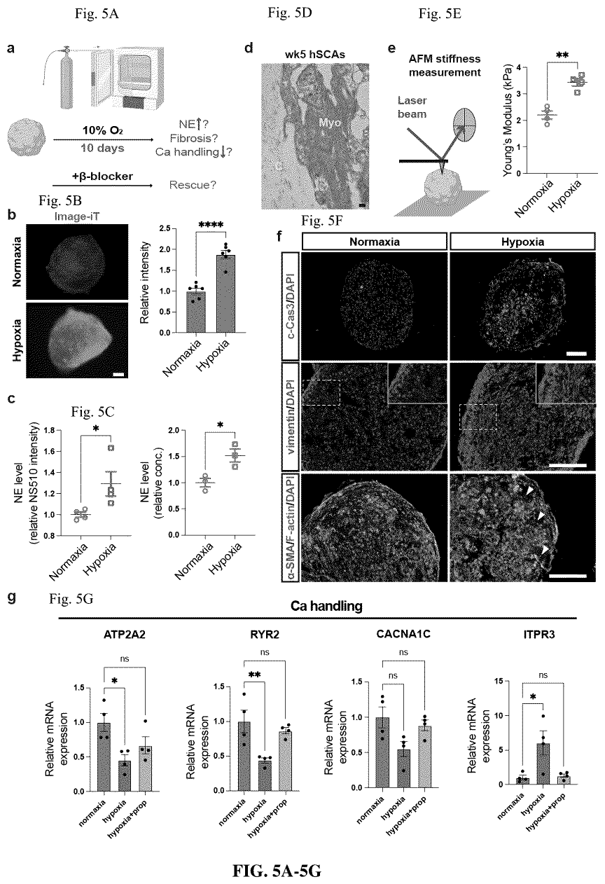

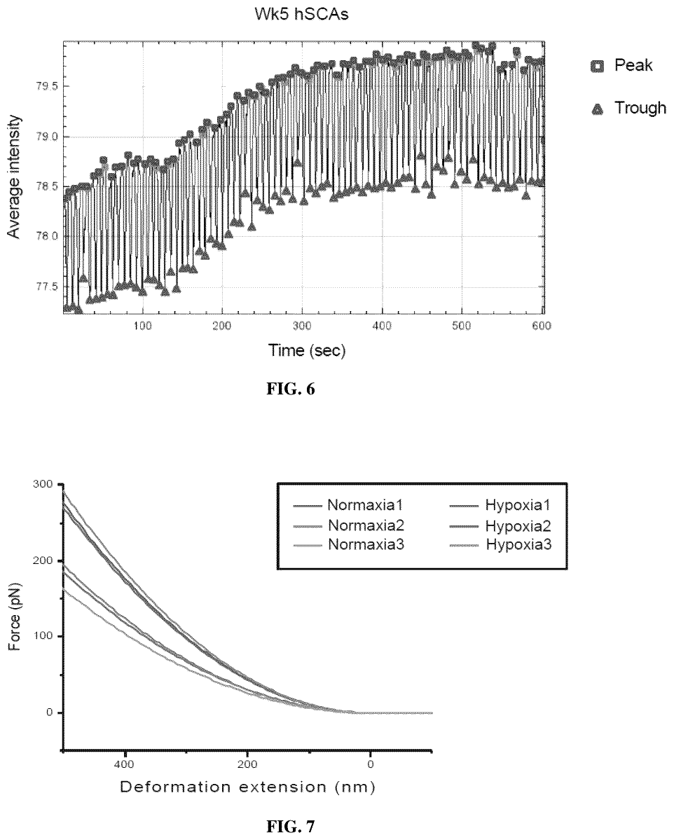

To test the effect of sympathetic input to cardiac function in the assembloids in a diseased state, a strategy was developed to model myocardial infarction. A model of myocardial infarction in cardiac organoids has been described⁵⁵. In this model, a moderate hypoxic environment (10% O₂) was applied, which prevented massive and sudden cell death in 3D cardiac microtissues and allowed the observation of the progression of infarction. In addition to hypoxia, exogenous NE was added to the cardiac organoids to mimic the effects of increased sympathetic tone to the heart. The combined treatments resulted in infarction of the cardiac organoids at a state similar to mice with myocardial injury⁵⁵. Here, this infraction model was utilized, and the conditions applied to the disclosed hSCAs. hSCAs were subjected to 10% O₂ for 10 days without exogenous NE and whether the symNs in the organoids become responsive to the hypoxic stress (FIG. 5A) was examined. After low oxygen treatment, hypoxic hSCAs were recognized by Image-iT™ Hypoxia Reagent compared to normoxic controls (FIG. 5B). The hypoxic stress stimulated overproduction of endogenous NE from symNs in hSCAs, measured via the NS510 probe and ELISA (FIG. 5C). Extracellular matrix in the heart, such as collagen, supports heart structural organization and development, but is also the component that forms the scar tissue in a damaged heart⁵⁶⁻⁵⁸. TEM imaging identified collagen in hSCAs (FIG. 5D). It has been shown that aberrant ECM accumulation and imbalanced degradation in the heart leads to the increased stiffness in cardiac fibrosis⁵⁶⁻⁵⁸. To evaluate this stiffening effect in cardiac infarction mimicking hSCAs, atomic force microscopy (AFM) was used to measure the stiffness and compare it between normoxia or hypoxia-treated hSCAs (FIG. 5E and FIG. 7). Hypoxic hSCAs showed increased stiffness compared to hSCAs in the normoxic environment (FIG. 5E). To further support the findings of fibrosis, hSCAs were stained for cleaved caspase-3 (c-Cas3) to detect apoptotic cells in the organoids. Compared to normoxic controls, hypoxic hSCAs displayed high amounts of c-Cas3⁺ cells in the center of the assembloids, likely due to the deficiency in oxygen supply (FIG. 5F). In addition, using the cardiac fibroblast marker vimentin that labels fibrotic tissue, increased vimentin signals on the outskirt of the hypoxic hSCAs were observed. Vimentin⁺ cells in that area showed elongated and elastic morphology, which is a typical feature of fibrotic tissues⁵⁵ (FIG. 5F). The fibrotic cardiac scar tissues in hypoxic hSCAs was further confirmed by assessing the colocalizing level of α-SMA and F-actin⁵⁵. In the outskirt area, hypoxic hSCAs showed higher colocalization of α-SMA⁺/F-actin⁺ cells compared to normoxic hSCAs (FIG. 5F). RT-qPCR analysis showed that the expression of Ca²⁺ handling genes were altered in hypoxic hSCAs, with a pattern similar to previously shown55, indicating that Ca²⁺ handling capacity was impaired in hypoxic hSCAs (FIG. 5G).

hSCAs present a powerful tool to screen for drugs for heart failure. In the clinic, β-blockers (propranolol, a BAR antagonist), which blocks the excitatory effects of NE to the heart are prescribed for heart failure and to prevent a second infarction^{59,60}. Thus, to further evaluate the potential of hSCAs for future cardiotoxicity studies, hypoxic hSCAs were treated with propranolol (FIG. 5A). When treating hSCAs with propranolol (1 μM) in addition to low oxygen, the impaired expression of Ca²⁺ handling genes were rescued (FIG. 5F). These results indicate that hSCAs possess a functional symN-cardiac tissue axis, which is responsive to environmental inputs at both healthy and diseased states and may be used for cardiotoxicity screening.

### DISCUSSION

Organoids are self-organizing 3D cell cultures that mimic some of the cellular, structural, and functional complexity of the native organ in vitro. They provide valuable insights to understand the structure-function relationship of human organs that 2D models cannot achieve, such as brain lobe structure, renal pyramid function and heart spatial patterning. Assembloids result from the integration of multiple organoids or combination of organoids with other cell types¹⁻³. They have the advantage of facilitating the study of interaction of tissues that may not normally develop from the same progenitor, for example forebrain and hindbrain¹ or vasculature and brain tissue⁶¹. A major outstanding issue in the organoid/assembloid field is the lack of innervation of peripheral tissue organoids. This is despite the fact, that almost all organs outside the brain are innervated by the peripheral nervous system, and that this neural regulation is important to the development, integrity, and function of organs.

Few innervated organoids have been reported to date; however, the common disadvantage of those reports is that they cannot be easily adapted to other organ type organoids or are technically difficult to reproduce by researchers. Workman et al. mixed specified hPSC-derived vagal NC cells with developing gut tube organoids and created an intestinal organoid with enteric neuron innervation⁹. However, enteric neurons are specific to the GI tract and do not innervate other organs, thus, making this less universal as an innervation strategy for many different organoid types. Schneider et al. created a 3D bioengineered hPSC-derived CM model with autonomic innervation using hPSC-derived autonomic organoids¹². CMs in this model were plated in circular form on dynamic stretch devices, which achieved advanced maturation of cardiac tissues. Autonomic neural organoids were then stuck into the ring of the engineered cardiac organoids for innervation and regulation thereof. However, these assembloids did not mimic the heart structures (ventricle/atrial patterning, heart cavity, for instance) and required specific instrumentation for their analysis. Innervated muscle models, where motor neurons connect to skeletal muscle, have been achieved using the one-pot differentiation strategy from the neuromesodermal progenitors¹⁰. This demonstrated the possibility to study complicated and anatomically distal functional coupling in organoids. However, not many organs develop their cell types and innervation from a common progenitor, such as the neuromesodermal progenitor, thus this strategy is not easily transferrable to other organoids. The assembloid technology, on the other hand, presents an ideal modular platform for individual organoid components, that is suitable for innervation of organoids^{2,3}.

Provided herein is a strategy to address the need of a method to innervate any organ type organoid relatively easily with symNs. As an example, cardiac organoids were created that are innervated by symNs. The assemble method is simple, easy to reproduce, and relatively low-cost, and does not require bioengineering or special instrumentation. Assembloids are created in a modular way, so that researchers can adapt them to their needs. For example, one could use the symNs to innervate other organoids, such as lung, kidney, or liver. Or one could replace the symNs and innervate the cardiac organoids with parasympathetic neurons (dx.doi.org/10.2139/ssrn.4318816) or sensory neurons^{62,63}.

Organoids or assembloids preferably fulfil certain criteria to be useful^{3,28,40} (1) 3D cultures. Several groups have generated 2D co-culture models of symN-innervated CMs in recent years. In 2016, Oh et al.

demonstrated a functional coupling 2D co-culture model, using hPSC-derived symNs and mouse neonatal ventricular myocytes, and showed that the beating rate of CMs can be regulated by symNs, and can be manipulated with nicotine or optogenetic stimulation⁵⁰. Later, Larsen et al. established a 2D co-culture system of neonatal ventricular myocytes and sympathetic stellate neurons from control Wistar Kyoto (WKY) and pro-hypertensive (SHR) rats. They found that hypertensive symNs were able to induce hypertensive phenotypes in healthy CMs, while healthy symNs rescued the hypertensive state in CMs⁵¹. In 2020, Winbo et al. performed the 2D co-culture model using symNs and CMs both derived from hPSC, which also displayed functional coupling regulation of CMs through symNs, inducible by nicotine⁴⁹. In 2022, it was described that symNs derived from iPSCs from the genetic autonomic disorder Familial Dysautonomia (FD) were hyperactive and in 2D co-cultures increased hPSC-derived CM beating²⁹. Adding the option of a 3D organoid model to this toolset for disease modeling will increase its power for discovery of disease mechanisms and drug discovery. The disclosed hSCAs are cultured in 3D throughout their generation and maturation stages (FIG. 1A-1N). (2) Self-organization from stem or progenitor cells. The disclosed hSCAs are assembled by mixing day 14 symNblasts and day 7 cardiac progenitors (FIG. 1A-1N). Reports^{29,30,35,36} show that these progenitors will form fully differentiated and functional symNs or cardiomyocytes upon continued 2D culture. Furthermore, these symNs have been employed to modeled autonomic dysfunctions in Familial Dysautonomia, within the SARS-COV-2 infection milieu, and under diabetic hyperglycemia conditions^{29,64,65}. (3) Contain multiple cell types that mimic the native organ. Here it is shown that the hSCAs contain multiple cardiac and symN lineage cell types. The human heart contains multiple cardiac lineages in addition to CMs, such as endothelial cells, smooth muscle cells, and cardiac fibroblast^{33,34}. The disclosed hSCAs contained mature cardiac muscle fibers, T-tubules, cardiac fibroblasts, epicardial and endocardial layers, as well as atrial and ventricular CMs (FIG. 2B-2D and 2H-2I, and 5F). (4) Mimicking some structural and functional features of the native organ. hSCAs contained CM-innervating functional symNs, in which NE synthesis was detected (FIG. 3A-3E). The functional coupling between symNs and CMs was observed by Ca²⁺ imaging and could be manipulated by nicotine and optogenetic stimulation (FIG. 3F-3G).

It is becoming more and more clear that tissue innervation is important for proper development, maturation and even repair of most organ tissues⁶⁶⁻⁷¹ Accordingly, symN innervation of the heart plays an important role in development and reciprocal maturation of the tissues. In 2015, Kreipke et al. used the neurotoxin 6-hydroxydopamine (6-OHDA) to induce symN lesions in neonatal mouse hearts and found that the proliferation of CMs was increased, indicating disrupted cell cycle withdrawal due to the lack of symN innervation²². In 2021, Tampakakis et al. demonstrated similar results using a smooth muscle-specific NGF deprivation mouse model, which resulted in heart-specific symN depletion in embryonic hearts, as well as increased CM proliferation²³. Interestingly, while both studies showed increased proliferation of CMs due to the absence of symN innervation, the model by Kreipke et al. showed decreased heart size after symN depletion, whereas Tampakakis et al. showed enlarged heart size. This might be due to the difference in CM density (unchanged in Kreipke et al. and increased in Tampakakis et al.) in each model and the unknown effect of symN signaling to other cell types that form the heart mass^{22,23}. Additionally, it is believed that such regulation on developmental cardiac hypertrophy by symNs is mediated by NE and adrenergic signaling^{72,73}. Indeed, in 2022, Kowalski et al. co-cultured mouse primary symNs with hPSC-derived CMs and showed that with symN innervation, the mature cardiac gene expression and functional cardiac activity were improved⁵³. However, they also found that treating hPSC-derived CMs alone with isoproterenol, a β-adrenergic receptor agonist, was not sufficient to induce such maturity improvement without physical connection with symNs⁵³, implying undiscovered mechanisms in the symN-heart axis, which may also account for the reason of different heart sizes observed in the models above (PO symN depletion in Kreipke et al., in which embryonic innervation remained, versus symN null in the heart in Tampakakis et al.). The here described hSCAs, therefore, might be an ideal model to assess development and reciprocal maturation of symNs and CMs in cultures. Using hSCAs, early heart development was modeled. Since the exact effect of NE on CMs is not fully clarified^{22,23,53}, α- and β-adrenergic receptor antagonist LAB was used to fully block the entire downstream target of NE (FIG. 4B). In this model, hSCA size and cell number were not altered in LAB-treated organoids compared to control (FIG. 4C). Additionally, genes for cardiac maturation decreased upon LAB treatment, indicating the importance of functional symN signaling for heart development and maturation.

Finally, the organoid technology has become an important tool for disease modeling approaches^{4-8,31,32,44,74}. In line with such studies, hSCAs were employed to model the hypoxia-induced cardiac infraction (FIG. 5A). The endogenous NE crisis was successfully recapitulated in hSCAs from symNs upon hypoxic stress, which caused cardiac fibrosis that was rescued by treatment with the β blocker propranolol along with the hypoxic stress (FIG. 5B-5G). It can be considered that in the whole organism, other NE releasing tissues, such as adrenal chromaffin cells, can also contribute to the NE crisis, and during heart failure, the inflammation response was not recapitulated in hSCAs, due the lack of immune cells in the organoids. Such challenges may be addressed by incorporating more distal tissues, such as chromaffin or immune cells into the assembloid.

Zeltner N, Wu H F, Saito-Diaz K, Sun X, Song M, Saini T, Grant C, James C, Thomas K, Abate Y, Howerth E, Kner P, Xu B. A modular platform to generate functional sympathetic neuron-innervated heart assembloids. Res Sq [Preprint]. 2024 Mar. 21: rs.3.rs-3894397. doi: 10.21203/rs.3.rs-3894397/v1. PMID: 38562819; PMCID: PMC10984094, is specifically incorporate by reference herein in its entirety.

TABLE 1

| Antibodies | Brand | Catelog | Host | Dilution |
|---|---|---|---|---|
| α-actinin | Sigma | A7811 | Mouse | 1:1000 |
| | | | IgG1 | |
| αSMA | Sigma | A5228 | mIgG2a | 1:1000 |
| c-Cas3 | Cell Signaling | 9661 | Rabbit | 1:100 |
| cTnT | Proteintech | 15513-1-AP | Rabbit | 1:200 |
| DAPI | Sigma | D9542 | — | 1:1000 |
| MLC-2a | Synaptic | 311011 | Mouse | 1:200 |
| | Systems | | IgG1 | |
| MLC-2v | Proteintech | 10906-1-AP | Rabbit | 1:200 |
| NFATC1 | Proteintech | 66963-1-Ig | Mouse | 1:200 |
| | | | IgG2b | |
| PRPH | Santa Cruz | SC- | Mouse | 1:500 |
| | Biotechnology | 377093/H0112 | IgG2a | |
| SYP | Santa Cruz | sc-17750 | Mouse | 1:200 |
| | Biotechnology | | | |
| TH | Pel-Freez | P40101- 150 | Rabbit | 1:500 |
| Vimentin | Abcam | ab92547 | Rabbit | 1:100 |
| VMAT2 | R&D Systems | MAB8327 | Mouse | 1:50 |
| | | | IgG1 | |
| WT1 | Abcam | ab89901 | Rabbit | 1:100 |

### REFERENCES

1. Makrygianni, E. A., and Chrousos, G. P. (2021). From Brain Organoids to Networking Assembloids: Implications for Neuroendocrinology and Stress Medicine. Front Physiol 12, 621970. 10.3389/fphys.2021.621970.
2. Vogt, N. (2021). Assembloids. Nat Methods 18, 27. 10.1038/s41592-020-01026-x.
3. Kanton, S., and Pasca, S. P. (2022). Human assembloids. Development 149. 10.1242/dev.201120.
4. Cala, G., Sina, B., De Coppi, P., Giobbe, G. G., and Gerli, M. F. M. (2023). Primary human organoids models: Current progress and key milestones. Front Bioeng Biotechnol 11, 1058970. 10.3389/fbioe.2023.1058970.
5. Corsini, N. S., and Knoblich, J. A. (2022). Human organoids: New strategies and methods for analyzing human development and disease. Cell 185, 2756-2769. 10.1016/j.cell.2022.06.051.
6. Kim, J., Koo, B. K., and Knoblich, J. A. (2020). Human organoids: model systems for human biology and medicine. Nat Rev Mol Cell Biol 21, 571-584. 10.1038/s41580-020-0259-3.
7. Lehmann, R., Lee, C. M., Shugart, E. C., Benedetti, M., Charo, R. A., Gartner, Z., Hogan, B., Knoblich, J., Nelson, C. M., and Wilson, K. M. (2019). Human organoids: a new dimension in cell biology. Mol Biol Cell 30, 1129-1137. 10.1091/mbc.E19-03-0135.
8. Tang, X. Y., Wu, S., Wang, D., Chu, C., Hong, Y., Tao, M., Hu, H., Xu, M., Guo, X., and Liu, Y. (2022). Human organoids in basic research and clinical applications. Signal Transduct Target Ther 7, 168. 10.1038/s41392-022-01024-9.
9. Workman, M. J., Mahe, M. M., Trisno, S., Poling, H. M., Watson, C. L., Sundaram, N., Chang, C. F., Schiesser, J., Aubert, P., Stanley, E. G., et al. (2017). Engineered human pluripotent-stem-cell-derived intestinal tissues with a functional enteric nervous system. Nat Med 23, 49-59. 10.1038/nm.4233.
10. Faustino Martins, J. M., Fischer, C., Urzi, A., Vidal, R., Kunz, S., Ruffault, P. L., Kabuss, L., Hube, I., Gazzerro, E., Birchmeier, C., et al. (2020). Self-Organizing 3D Human Trunk Neuromuscular Organoids. Cell Stem Cell 26, 172-186 e176. 10.1016/j.stem.2019.12.007.
11. Zhang, X. S., Xie, G., Ma, H., Ding, S., Wu, Y. X., Fei, Y., Cheng, Q., Huang, Y., and Wang, Y. (2023). Highly reproducible and cost-effective one-pot organoid differentiation using a novel platform based on PF-127 triggered spheroid assembly. Biofabrication 15. 10.1088/1758-5090/acee21.
12. Schneider, L. V., Guobin, B., Methi, A., Jensen, O., Schmoll, K. A., Setya, M. G., Sakib, S., Fahud, A. L., Brockmöller, J., Fischer, A., et al. (2023). Bioengineering of a human innervated cardiac muscle model. 2023.2008.2018.552653. 10.1101/2023.08.18.552653% J bioRxiv.
13. Tsao, C. W., Aday, A. W., Almarzooq, Z. I., Anderson, C. A. M., Arora, P., Avery, C. L., Baker-Smith, C. M., Beaton, A. Z., Boehme, A. K., Buxton, A. E., et al. (2023). Heart Disease and Stroke Statistics-2023 Update: A Report From the American Heart Association. Circulation 147, e93-e621. 10.1161/CIR.0000000000001123.
14. McCorry, L. K. (2007). Physiology of the autonomic nervous system. Am J Pharm Educ 71, 78. 10.5688/aj710478.
15. Schlaich, M. P., Lambert, E., Kaye, D. M., Krozowski, Z., Campbell, D. J., Lambert, G., Hastings, J., Aggarwal, A., and Esler, M. D. (2004). Sympathetic augmentation in hypertension: role of nerve firing, norepinephrine reuptake, and Angiotensin neuromodulation. Hypertension 43, 169-175. 10.1161/01.HYP.0000103160.35395.9E.
16. Niedermaier, O. N., Smith, M. L., Beightol, L. A., Zukowska-Grojec, Z., Goldstein, D. S., and Eckberg, D. L. (1993). Influence of cigarette smoking on human autonomic function. Circulation 88, 562-571. 10.1161/01.cir.88.2.562.
17. Price, R. W. (1977). Viral infections of the autonomic nervous system and its target organs: pathogenetic mechanisms. Med Hypotheses 3, 33-36. 10.1016/0306-9877 (77) 90049-4.
18. Schlaich, M., Straznicky, N., Lambert, E., and Lambert, G. (2015). Metabolic syndrome: a sympathetic disease? Lancet Diabetes Endocrinol 3, 148-157. 10.1016/S2213-8587 (14) 70033-6.
19. van de Borne, P., Mark, A. L., Montano, N., Mion, D., and Somers, V. K. (1997). Effects of alcohol on sympathetic activity, hemodynamics, and chemoreflex sensitivity. Hypertension 29, 1278-1283. 10.1161/01.hyp.29.6.1278.
20. Scott-Solomon, E., Boehm, E., and Kuruvilla, R. (2021). The sympathetic nervous system in development and disease. Nat Rev Neurosci 22, 685-702. 10.1038/s41583-021-00523-y.
21. Herring, N., Kalla, M., and Paterson, D. J. (2019). The autonomic nervous system and cardiac arrhythmias: current concepts and emerging therapies. Nat Rev Cardiol 16, 707-726. 10.1038/s41569-019-0221-2.
22. Kreipke, R. E., and Birren, S. (2015). Innervating sympathetic neurons regulate heart size and the timing of cardiomyocyte cell cycle withdrawal. J Physiol 593, 5057-5073. 10.1113/JP270917.
23. Tampakakis, E., Gangrade, H., Glavaris, S., Htet, M., Murphy, S., Lin, B. L., Liu, T., Saberi, A., Miyamoto, M., Kowalski, W., et al. (2021). Heart neurons use clock genes to control myocyte proliferation. Sci Adv 7, eabh4181. 10.1126/sciadv.abh4181.
24. Fukuda, K., Kanazawa, H., Aizawa, Y., Ardell, J. L., and Shivkumar, K. (2015). Cardiac innervation and sudden cardiac death. Circ Res 116, 2005-2019. 10.1161/CIRCRESAHA.116.304679.
25. Huang, W. A., Boyle, N. G., and Vaseghi, M. (2017). Cardiac Innervation and the Autonomic Nervous System in Sudden Cardiac Death. Card Electrophysiol Clin 9, 665-679. 10.1016/j.ccep.2017.08.002.
26. Schwartz, P. J. (1976). Cardiac sympathetic innervation and the sudden infant death syndrome. A possible pathogenetic link. Am J Med 60, 167-172. 10.1016/0002-9343 (76) 90425-3.
27. Schwartz, P. J. (1990). Cardiac sympathetic innervation and the prevention of sudden death. Cardiologia 35, 51-54.
28. Marton, R. M., and Pasca, S. P. (2020). Organoid and Assembloid Technologies for Investigating Cellular Crosstalk in Human Brain Development and Disease. Trends Cell Biol 30, 133-143. 10.1016/j.tcb.2019.11.004.
29. Wu, H. F., Yu, W., Saito-Diaz, K., Huang, C. W., Carey, J., Lefcort, F., Hart, G. W., Liu, H. X., and Zeltner, N. (2022). Norepinephrine transporter defects lead to sympathetic hyperactivity in Familial Dysautonomia models. Nat Commun 13, 7032. 10.1038/s41467-022-34811-7.
30. Wu, H. F., and Zeltner, N. (2020). Efficient Differentiation of Postganglionic Sympathetic Neurons using Human Pluripotent Stem Cells under Feeder-free and Chemically Defined Culture Conditions. J Vis Exp. 10.3791/60843.
31. Kim, H., Kamm, R. D., Vunjak-Novakovic, G., and Wu, J. C. (2022). Progress in multicellular human cardiac organoids for clinical applications. Cell Stem Cell 29, 503-514. 10.1016/j.stem.2022.03.012.
32. Zhao, D., Lei, W., and Hu, S. (2021). Cardiac organoid-a promising perspective of preclinical model. Stem Cell Res Ther 12, 272. 10.1186/s13287-021-02340-7.
33. Banerjee, I., Fuseler, J. W., Price, R. L., Borg, T. K., and Baudino, T. A. (2007). Determination of cell types and numbers during cardiac development in the neonatal and adult rat and mouse. Am J Physiol Heart Circ Physiol 293, H1883-1891. 10.1152/ajpheart.00514.2007.
34. Litvinukova, M., Talavera-Lopez, C., Maatz, H., Reichart, D., Worth, C. L., Lindberg, E. L., Kanda, M., Polanski, K., Heinig, M., Lee, M., et al. (2020). Cells of the adult human heart. Nature 588, 466-472. 10.1038/s41586-020-2797-4.
35. Lin, Y., Linask, K. L., Mallon, B., Johnson, K., Klein, M., Beers, J., Xie, W., Du, Y., Liu, C., Lai, Y., et al. (2017). Heparin Promotes Cardiac Differentiation of Human Pluripotent Stem Cells in Chemically Defined Albumin-Free Medium, Enabling Consistent Manufacture of Cardiomyocytes. Stem Cells Transl Med 6, 527-538. 10.5966/sctm.2015-0428.
36. Lin, Y., and Zou, J. (2020). Differentiation of Cardiomyocytes from Human Pluripotent Stem Cells in Fully Chemically Defined Conditions. STAR Protoc 1. 10.1016/j.xpro.2020.100015.
37. Amini, H., Rezaie, J., Vosoughi, A., Rahbarghazi, R., and Nouri, M. (2017). Cardiac progenitor cells application in cardiovascular disease. J Cardiovasc Thorac Res 9, 127-132. 10.15171/jcvtr.2017.22.
38. Brade, T., Pane, L. S., Moretti, A., Chien, K. R., and Laugwitz, K. L. (2013). Embryonic heart progenitors and cardiogenesis. Cold Spring Harb Perspect Med 3, a013847. 10.1101/cshperspect.a013847.
39. Achanta, S., Gorky, J., Leung, C., Moss, A., Robbins, S., Eisenman, L., Chen, J., Tappan, S., Heal, M., Farahani, N., et al. (2020). A Comprehensive Integrated Anatomical and Molecular Atlas of Rat Intrinsic Cardiac Nervous System. iScience 23, 101140. 10.1016/j.isci.2020.101140.
40. Pasca, S. P., Arlotta, P., Bateup, H. S., Camp, J. G., Cappello, S., Gage, F. H., Knoblich, J. A., Kriegstein, A. R., Lancaster, M. A., Ming, G. L., et al. (2022). A nomenclature consensus for nervous system organoids and assembloids. Nature 609, 907-910. 10.1038/s41586-022-05219-6.
41. Guo, Y., and Pu, W. T. (2020). Cardiomyocyte Maturation: New Phase in Development. Circ Res 126, 1086-1106. 10.1161/CIRCRESAHA.119.315862.
42. Brette, F., and Orchard, C. (2003). T-tubule function in mammalian cardiac myocytes. Circ Res 92, 1182-1192. 10.1161/01.RES.0000074908.17214.FD.
43. Hong, T., and Shaw, R. M. (2017). Cardiac T-Tubule Microanatomy and Function. Physiol Rev 97, 227-252. 10.1152/physrev.00037.2015.
44. Zhao, Z., Chen, X., Dowbaj, A. M., Sljukic, A., Bratlie, K., Lin, L., Fong, E. L. S., Balachander, G. M., Chen, Z., Soragni, A., et al. (2022). Organoids. Nat Rev Methods Primers 2. 10.1038/s43586-022-00174-y.
45. Rockel, A. F., Wagner, N., Spenger, P., Ergun, S., and Worsdorfer, P. (2023). Neuro-mesodermal assembloids recapitulate aspects of peripheral nervous system development in vitro. Stem Cell Reports 18, 1155-1165. 10.1016/j.stemcr.2023.03.012.
46. Zaglia, T., and Mongillo, M. (2017). Cardiac sympathetic innervation, from a different point of (re) view. J Physiol 595, 3919-3930. 10.1113/JP273120.
47. Foulon, P., and De Backer, D. (2018). The hemodynamic effects of norepinephrine: far more than an increase in blood pressure! Ann Transl Med 6, S25. 10.21037/atm.2018.09.27.
48. Zhang, L., Liu, X. A., Gillis, K. D., and Glass, T. E. (2019). A High-Affinity Fluorescent Sensor for Catecholamine: Application to Monitoring Norepinephrine Exocytosis. Angew Chem Int Ed Engl 58, 7611-7614. 10.1002/anie.201810919.
49 Winbo, A., Ramanan, S., Eugster, E., Jovinge, S., Skinner, J. R., and Montgomery, J. M. (2020). Functional coculture of sympathetic neurons and cardiomyocytes derived from human-induced pluripotent stem cells. Am J Physiol Heart Circ Physiol 319, H927-H937. 10.1152/ajpheart.00546.2020.
50. Oh, Y., Cho, G. S., Li, Z., Hong, I., Zhu, R., Kim, M. J., Kim, Y. J., Tampakakis, E., Tung, L., Huganir, R., et al. (2016). Functional Coupling with Cardiac Muscle Promotes Maturation of hPSC-Derived Sympathetic Neurons. Cell Stem Cell 19, 95-106. 10.1016/j.stem.2016.05.002.
51. Larsen, H. E., Lefkimmiatis, K., and Paterson, D. J. (2016). Sympathetic neurons are a powerful driver of myocyte function in cardiovascular disease. Sci Rep 6, 38898. 10.1038/srep38898.
52. Abilez, O. J. (2012). Cardiac optogenetics. Annu Int Conf IEEE Eng Med Biol Soc 2012, 1386-1389. 10.1109/EMBC.2012.6346197.
53. Kowalski, W. J., Garcia-Pak, I. H., Li, W., Uosaki, H., Tampakakis, E., Zou, J., Lin, Y., Patterson, K., Kwon, C., and Mukouyama, Y. S. (2022). Sympathetic Neurons Regulate Cardiomyocyte Maturation in Culture. Front Cell Dev Biol 10, 850645. 10.3389/fcell.2022.850645.
54 Poon, E. N., Luo, X. L., Webb, S. E., Yan, B., Zhao, R., Wu, S. C. M., Yang, Y., Zhang, P., Bai, H., Shao, J., et al. (2020). The cell surface marker CD36 selectively identifies matured, mitochondria-rich hPSC-cardiomyocytes. Cell Res 30, 626-629. 10.1038/s41422-020-0292-y.
55. Richards, D. J., Li, Y., Kerr, C. M., Yao, J., Beeson, G. C., Coyle, R. C., Chen, X., Jia, J., Damon, B., Wilson, R., et al. (2020). Human cardiac organoids for the modelling of myocardial infarction and drug cardiotoxicity. Nat Biomed Eng 4, 446-462. 10.1038/s41551-020-0539-4.
56. Hinderer, S., and Schenke-Layland, K. (2019). Cardiac fibrosis-A short review of causes and therapeutic strategies. Adv Drug Deliv Rev 146, 77-82. 10.1016/j.addr.2019.05.011.
57. Silva, A. C., Pereira, C., Fonseca, A., Pinto-do, O. P., and Nascimento, D. S. (2020). Bearing My Heart: The Role of Extracellular Matrix on Cardiac Development, Homeostasis, and Injury Response. Front Cell Dev Biol 8, 621644. 10.3389/fcell.2020.621644.
58. Weber, K. T. (1997). Extracellular matrix remodeling in heart failure: a role for de novo angiotensin II generation. Circulation 96, 4065-4082. 10.1161/01.cir.96.11.4065.
59. Chavey, W. E., 2nd (2000). The importance of beta blockers in the treatment of heart failure. Am Fam Physician 62, 2453-2462.
60. Gheorghiade, M., Colucci, W. S., and Swedberg, K. (2003). Beta-blockers in chronic heart failure. Circulation 107, 1570-1575. 10.1161/01.CIR.0000065187.80707.18.
61. Song, L., Yuan, X., Jones, Z., Griffin, K., Zhou, Y., Ma, T., and Li, Y. (2019). Assembly of Human Stem Cell-Derived Cortical Spheroids and Vascular Spheroids to Model 3-D Brain-like Tissues. Sci Rep 9, 5977. 10.1038/s41598-019-42439-9.
62. Saito-Diaz, K., Street, J. R., Ulrichs, H., and Zeltner, N. (2021). Derivation of Peripheral Nociceptive, Mechanoreceptive, and Proprioceptive Sensory Neurons from the same Culture of Human Pluripotent Stem Cells. Stem Cell Reports 16, 446-457. 10.1016/j.stemcr.2021.01.001.
63. Saito-Diaz, K., and Zeltner, N. (2022). A protocol to differentiate nociceptors, mechanoreceptors, and proprioceptors from human pluripotent stem cells. STAR Protoc 3, 101187. 10.1016/j.xpro.2022.101187.
64. Wu, H. F., Huang, C. W., Art, J., Liu, H. X., Hart, G. W., and Zeltner, N. (2023). O-GlcNAcylation is crucial for sympathetic neuron development, maintenance, functionality and contributes to peripheral neuropathy. Front Neurosci 17, 1137847. 10.3389/fnins.2023.1137847.
65. Wu, H. F., Huang, C. W., Daga, K. R., Marklein, R. A., Ivanova, N., and Zeltner, N. (2022). Human pluripotent stem cell-derived functional sympathetic neurons express ACE2 and RAAS components: a framework for studying the effect of COVID-19 on sympathetic responsiveness. Clin Auton Res 32, 59-63. 10.1007/s10286-021-00850-1.
66. Borden, P., Houtz, J., Leach, S. D., and Kuruvilla, R. (2013). Sympathetic innervation during development is necessary for pancreatic islet architecture and functional maturation. Cell Rep 4, 287-301. 10.1016/j.celrep.2013.06.019.
67. Das, S., Gordian-Velez, W. J., Ledebur, H. C., Mourkioti, F., Rompolas, P., Chen, H. I., Serruya, M. D., and Cullen, D. K. (2020). Innervation: the missing link for biofabricated tissues and organs. NPJ Regen Med 5, 11. 10.1038/s41536-020-0096-1.
68. Honeycutt, S. E., N′Guetta, P. Y., and O'Brien, L. L. (2022). Innervation in organogenesis. Curr Top Dev Biol 148, 195-235. 10.1016/bs.ctdb.2022.02.004.
69. Revah, O., Gore, F., Kelley, K. W., Andersen, J., Sakai, N., Chen, X., Li, M. Y., Birey, F., Yang, X., Saw, N. L., et al. (2022). Maturation and circuit integration of transplanted human cortical organoids. Nature 610, 319-326. 10.1038/s41586-022-05277-w.
70. Wada, H., Dambly-Chaudiere, C., Kawakami, K., and Ghysen, A. (2013). Innervation is required for sense organ development in the lateral line system of adult zebrafish. Proc Natl Acad Sci USA 110, 5659-5664. 10.1073/pnas.1214004110.
71. White, I. A., Gordon, J., Balkan, W., and Hare, J. M. (2015). Sympathetic Reinnervation Is Required for Mammalian Cardiac Regeneration. Circ Res 117, 990-994. 10.1161/CIRCRESAHA.115.307465.
72. Engelhardt, S., Hein, L., Wiesmann, F., and Lohse, M. J. (1999). Progressive hypertrophy and heart failure in beta1-adrenergic receptor transgenic mice. Proc Natl Acad Sci USA 96, 7059-7064. 10.1073/pnas.96.12.7059.
73. Liu, H., Zhang, C. H., Ammanamanchi, N., Suresh, S., Lewarchik, C., Rao, K., Uys, G. M., Han, L., Abrial, M., Yimlamai, D., et al. (2019). Control of cytokinesis by beta-adrenergic receptors indicates an approach for regulating cardiomyocyte endowment. Sci Transl Med 11. 10.1126/scitranslmed.aaw6419.
74. Huch, M., Knoblich, J. A., Lutolf, M. P., and Martinez-Arias, A. (2017). The hope and the hype of organoid research. Development 144, 938-941. 10.1242/dev.150201.
75. Liu, Y., Song, M., Liu, B., and Kner, P. (2023). Single objective light sheet microscopy with multi-direction excitation and structured light (Conference Presentation) (SPIE).
76 Mollenhauer, H. H. (1964). Plastic Embedding Mixtures for Use in Electron Microscopy. Stain Technol 39, 111-114.
77. Bozzola, J. J. (2014). Conventional specimen preparation techniques for transmission electron microscopy of cultured cells. Methods Mol Biol 1117, 1-19. 10.1007/978-1-62703-776-1_1.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of making assembloids comprising combining dissociated neuron progenitor cells with dissociated tissue progenitor cells and culturing them under free floating 3D culture conditions suitable for the neuron progenitor cells and tissue progenitor cells to form one or more assembloids.

2. The method of claim 1, wherein the assembloids are cultured under suitable conditions and duration for the neuron progenitor cells and tissue progenitor cells to mature.

3. The method of claim 2, wherein maturation comprises expression of one or more markers; presence of one or more structures and/or ultrastructures; and/or one or more functionalities consistent with the corresponding mature tissue or system in vivo.

4. The method of claim 1, wherein the method is carried out free from special instruments and/or scaffolds, optionally wherein the special instrument and/or scaffold is or includes a bioprinter, hydrogel, and/or organ-mimicking scaffold.

5. The method of claim 1, wherein the neuron progenitor cells and tissue progenitor cells are mixed under movement or agitation, optionally using a shaker optionally wherein the shaker is an orbital shaker.

6. The method of claim 1, wherein the neuron progenitor cells and tissue progenitor cells, and/or assembloids are cultured under movement or agitation, optionally using a shaker optionally wherein the shaker is an orbital shaker.

7. The method of claim 1, wherein the neuron progenitor cells are sympathetic neuron progenitor cells, parasympathetic neuron progenitor cells, and/or sensory neuron progenitor cells, optionally wherein the sensory neuron progenitor cells are nociceptors, mechanoreceptors, and/or proprioceptors.

8. The method of claim 1, wherein the tissue progenitor cells are heart, lung, kidney, liver, salivary gland, skin, and/or gastro-intestinal progenitor cells.

9. The method of claim 1, wherein the neuron progenitor cells are symNblast sympathetic neuron progenitor cells prepared from human pluripotent stem cells.

10. The method of claim 1, wherein the tissue progenitor cells are day 7 cardiomyocytes prepared from human pluripotent stem cells.

11. An assembloid formed according to the method of claim 1.

12. A 3D assembloid comprising neurons innervating a second or more tissues.

13. The assembloid of claim 11, wherein the neurons comprise sympathetic neurons, parasympathetic neurons, and/or sensory neurons, optionally wherein the sensory neurons are nociceptors, mechanoreceptors, and/or proprioceptors.

14. The assembloid of claim 11, wherein the second or more tissues comprise heart, lung, kidney, liver, salivary gland, skin, and/or gastro-intestinal cells.

15. An assembloid comprising sympathetic neurons and cardiac cells.

16. A composition comprising the assembloid of claim 11.

17. The composition of claim 16 comprising culture media.

18. Conditioned media formed by culturing assembloid(s) of claim 11.

19. A method of determining the effect of a compound comprising measuring one or more characteristic(s) of cultured assembloid(s) of claim 11 a first time, contacting the assembloid(s) with the compound, and measuring the characteristic(s) of the cultured assembloid(s) a second time.

20. The method of claim 19, wherein a plurality of compounds are separately contacted with a plurality of separately cultured assembloids, optionally wherein the assembloids are separately cultured in a multiwell plate or dish.
